(19) United States
(12) Patent Application Publication
(10) Pub. No.: US 2011/0055947 A1
(43) Pub. Date: Mar. 3, 2011

(54) MELON SLIP HARVEST INDICATOR

(76) Inventors: PAUL CHUNG, Woodland, CA (US); Jeffrey Mills, Woodland, CA (US); Adam Wentzell, Woodland, CA (US)

(21) Appl. No.: 12/872,897

(22) Filed: Aug. 31, 2010

Related U.S. Application Data
(60) Provisional application No. 61/238,563, filed on Aug. 31, 2009.

(51) Int. Cl. A01H 1/04; A01H 5/00; A01H 5/10; A01H 5/12; A01H 5/06; A01H 5/08; C12N 5/04; C12N 15/82; C12Q 1/68; A01G 1/00

(52) U.S. Cl. 800/260; 800/309; 435/410; 800/278; 435/6; 47/58.1 R

(57) ABSTRACT

The invention provides a Cucumis melo var. inodorus melon of consistent harvest quality. One embodiment provides use of fruit separation from the stem at harvest maturity to indicate harvest maturity of Cucumis melo var. inodorus melons. In specific embodiments, the fruit separation from the stem at harvest maturity indicator demonstrates harvest maturity on a per melon basis, can be scored visually or by force necessary to remove the melon from the plant, and reduces wounding damage upon separation of the fruit from the vine. Significantly, the invention provides for consistent harvest maturity of Cucumis melo var. inodorus melons that retain flesh firmness at the time of harvest and upon transport to market.

### CROSS-REFERENCE TO RELATED APPLICATIONS

This application claims the priority of U.S. Provisional Appl. Ser. No. 61/238,563, filed Aug. 31, 2009, the entire disclosure of which is incorporated herein by reference.

### FIELD OF THE INVENTION

The field of the present invention is melon breeding and the genetic improvement of melons. More specifically, this application is related to Cucumis melo var. inodorus melon seeds and plants for the production of consistent quality melon fruit that separate from the vine at harvest maturity when flesh firmness and sugar levels are both elevated.

### BACKGROUND OF THE INVENTION

Melons such as honeydews are typically harvested and then may spend hours to days or several weeks in transit before arriving at a marketplace where they are offered for commercial sale. This transport period may vary depending on the proximity of field to market and whether the melons are sold domestically or internationally. This requires many melons to be harvested at a time of maturity that allows for transport and flesh firmness while still yielding a product with high sugar levels.

Currently Cucumis melo var. inodorus melons such as honeydews do not exhibit a clear harvest indicator that growers could use to determine when melons would be ready to harvest and transport to market. In some varieties a color change (usually from green-white to creamy yellow) occurs at maturity, however, this color change is most clearly discernable when melons are overripe with soft, watery flesh. Sugar levels can also be used as a harvest indicator but this is a destructive measurement requiring access to the interior of the melon. Since this can only be performed on a test sample of melons, this method of harvest maturity assessment can only be a rough measure of the level of maturity across a field. To date, it is difficult to harvest many melons at a consistent maturity level that still allows for transport to market.

When melons such as honeydews are picked too early, the Brix level is too low, as sugar is a major contributor to melon flavor and sugar accumulation in the flesh does not continue after the fruit has been separated from the vine. When fruit are picked too late, the flesh is watery and becoming soft and fruit does not hold up during transport.

### SUMMARY OF THE INVENTION

In one aspect, the invention provides a Cucumis melo var. inodorus plant, wherein a melon from the plant exhibits separation from the stem at harvest maturity. The melon plant may be defined as of a commercially acceptable variety. In particular embodiments, the melon of the plant comprises flesh firmness from about 7 PSI to about 14 PSI, when measured by penetrometer with an 11 millimeter probe, at the time of melon separation from the stem. In another embodiment, the melon of the plant comprises a Brix content from about 10° Brix to about 17° Brix, when measured at melon separation from the stem. In certain embodiments, the Cucumis melo var. inodorus plant of the invention is hybrid or inbred. Seed and parts of the Cucumis melo var. inodorus plant of the present invention are also provided, for example, including a leaf, pollen, an ovule, a fruit, rootstock, a scion, and a cell. In a particular embodiment, the plant part is a fruit or melon. In still further embodiments, the Cucumis melo var. inodorus plant is of a melon market class selected from Piel de Sapo, Juan Canary (also known as and specifically including Jaune des Canaries and Amarillo), Earl's Type, Honeydew, Orange flesh honeydew, Hami Melon, Crenshaw and Casaba.

In another aspect of the invention, a Cucumis melo var. inodorus plant of the invention comprising an added heritable trait is provided. The heritable trait may comprise a genetic locus that is, for example, a dominant or recessive allele. In one embodiment of the invention, a Cucumis melo var. inodorus plant is defined as comprising a single locus conversion. In specific embodiments of the invention, an added genetic locus confers one or more traits such as, for example, herbicide tolerance, insect resistance, disease resistance, and modified carbohydrate metabolism. In further embodiments, the trait may be conferred by a naturally occurring gene introduced into the genome of the line by backcrossing, a natural or induced mutation, or a transgene introduced through genetic transformation techniques into the plant, a plant cell or a progenitor of any previous generation thereof. When introduced through transformation, a genetic locus may comprise one or more genes integrated at a single chromosomal location.

In another aspect of the invention, a tissue culture of regenerable cells of a Cucumis melo var. inodorus plant of the invention is provided. The regenerable cells in such tissue cultures may be derived from embryos, meristems, cotyledons, pollen, leaves, anthers, roots, root tips, pistil, flower, seed and stalks. Still further, the present invention provides Cucumis melo var. inodorus plants regenerated from a tissue culture of the invention.

In particular embodiments, the trait for separation from the stem at harvest maturity is controlled by genetic means for the expression of such a trait found in a plant selected from the group consisting of inbred line HDG39-2021AN, a sample of seed of which has been deposited under ATCC Accession No. PTA-10257; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

In further embodiments, a Cucumis melo var. inodorus plant of the invention is selected from the group consisting of inbred line HDG39-2021AN, a sample of seed of which has been deposited under ATCC Accession No. PTA-10257; HDG39-2022AN, a sample of seed of which has been deposited under ATCC Accession No. ______; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

In another aspect, the invention provides a Cucumis melo var. inodorus plant obtainable by crossing a first plant of the invention with a second plant, wherein the Cucumis melo var. inodorus plant produces melons that exhibit separation from the stem at harvest maturity. In one embodiment, the first plant is a plant of a variety selected from the group consisting of inbred line HDG39-2021AN, a sample of seed of which has been deposited under ATCC Accession No. PTA-10257; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

In yet another aspect, the invention provides a Cucumis melo var. inodorus melon that retains flesh firmness at the time of exhibition of slip, or fruit separation from the stem. In one embodiment, the melon has flesh firmness from about 7 PSI to about 14 PSI, when measured by penetrometer with an 11 millimeter probe, and no residual stem upon detachment from the plant. In another embodiment, the melon comprises a Brix content from about 10° Brix to about 17° Brix, when measured at melon separation from the stem.

The invention further provides methods for identifying a Cucumis melo var. inodorus melon at harvest maturity comprising detecting separation of the melon from the stem. In particular embodiments, detecting separation of the melon from the stem comprises mechanical detection, for instance detecting a reduction in resistance in melon separation from the stem, or comprises visual detection of fruit separation from the vine at the point of attachment.

In further aspects, the invention provides methods for producing a Cucumis melo var. inodorus of the invention. Such methods generally comprise (a) crossing a first Cucumis melo var. inodorus plant with a second melon plant capable of being crossed to said first Cucumis melo var. inodorus plant and that exhibits separation of a melon from the stem at harvest maturity; (b) selecting an F1 progeny that exhibits separation of the melon from the stem at harvest maturity; (c) crossing the selected F1 progeny with the first Cucumis melo var. inodorus plant to produce a backcross progeny; (d) selecting backcross progeny that exhibit separation of the melon from the stem at harvest maturity and comprise the physiological and morphological characteristics of the first Cucumis melo var. inodorus plant; and (e) repeating steps (c) and (d) three or more times to produce a selected fourth or higher backcross progeny plant that exhibits separation of the melon from the stem at harvest maturity. In one embodiment, the method further comprises (f) crossing the selected backcross progeny plant with a second Cucumis melo var. inodorus plant to produce seed of a hybrid progeny plant. In certain embodiments the second plant may be a plant other than a Cucumis melo var. inodorus plant. For instance, the second plant may be a plant of the line PI 414723.

In other aspects, the invention provides methods for vegetatively propagating a Cucumis melo var. inodorus of the invention. Such methods generally comprise (a) obtaining tissue capable of being propagated from a plant according to the invention; (b) cultivating said tissue to obtain proliferated shoots; and (c) rooting said proliferated shoots to obtain rooted plantlets. The method may additionally comprise growing plants from the rooted plantlets.

In yet another aspect of the invention, processes are provided for producing Cucumis melo var. inodorus seeds, plants and fruit, which processes generally comprise crossing a first parent Cucumis melo var. inodorus plant with a second parent plant that is capable of being crossed to the first parent Cucumis melo var. inodorus plant, wherein at least one of the first or second parent plants is a Cucumis melo var. inodorus of the invention. In such a method the first parent plant can be used as either a male or female parent in the crossing. These processes may be further exemplified as processes for preparing hybrid Cucumis melo var. inodorus seed or plants, wherein a first Cucumis melo var. inodorus plant of the invention is crossed with a second plant of a different, distinct line to provide a hybrid that has, as one of its parents, the Cucumis melo var. inodorus plant of the invention. In certain embodiments, the second plant may be of an inbred Cucumis melo var. inodorus line. In these processes, crossing will result in the production of seed. The seed production occurs regardless of whether the seed is collected or not.

In one embodiment of the invention, the first step in “crossing” comprises planting seeds of a first and second parent plant, often in proximity so that pollination will occur for example, mediated by insect vectors. Alternatively, pollen can be transferred manually. Where the plant is self-pollinated, pollination may occur without the need for direct human intervention other than plant cultivation.

A second step may comprise cultivating or growing the seeds of first and second parent plants into plants that bear flowers. A third step may comprise preventing self-pollination of the plants, such as by emasculating the male portions of flowers, (i.e., treating or manipulating the flowers to produce an emasculated parent plant). Self-incompatibility systems may also be used in some hybrid crops for the same purpose. Self-incompatible plants still shed viable pollen and can pollinate plants of other varieties but are incapable of pollinating themselves or other plants of the same line.

A fourth step for a hybrid cross may comprise cross-pollination between the first and second parent plants. Yet another step comprises harvesting the seeds from at least one of the parent plants. The harvested seed can be grown to produce a Cucumis melo var. inodorus or hybrid Cucumis melo var. inodorus.

In certain embodiments, the present invention provides a method of producing a Cucumis melo var. inodorus melon comprising: (a) obtaining a Cucumis melo var. inodorus plant of the invention, wherein the plant has been cultivated to maturity, and (b) collecting melon fruit from the plant.

In still yet another aspect of the invention, the genetic complement of the Cucumis melo var. inodorus plant of the invention is provided. The phrase “genetic complement” is used to refer to the aggregate of nucleotide sequences, the expression of which sequences defines the phenotype of, in the present case, a Cucumis melo var. inodorus plant, or a cell or tissue of that plant. A genetic complement thus represents the genetic makeup of a cell, tissue or plant, and a hybrid genetic complement represents the genetic make up of a hybrid cell, tissue or plant. The invention thus provides Cucumis melo var. inodorus plant cells that have a genetic complement in accordance with the Cucumis melo var. inodorus plant cells disclosed herein, and plants, seeds and plants containing such cells.

Plant genetic complements may be assessed by genetic marker profiles, and by the expression of phenotypic traits that are characteristic of the expression of the genetic complement, e.g., isozyme typing profiles. It is understood that plants of the present invention or a first generation progeny thereof could be identified by any of the many well known techniques such as, for example, Simple Sequence Length Polymorphisms (SSLPs) (Williams et al., 1990), Randomly Amplified Polymorphic DNAs (RAPDs), DNA Amplification Fingerprinting (DAF), Sequence Characterized Amplified Regions (SCARS), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., 1998).

In still yet another aspect, the present invention provides hybrid genetic complements, as represented by Cucumis melo var. inodorus cells, tissues, plants, and seeds, formed by the combination of a haploid genetic complement of a Cucumis melo var. inodorus plant of the invention with a haploid genetic complement of a second Cucumis melo var. inodorus plant, preferably, another, distinct Cucumis melo var. inodorus plant. In another aspect, the present invention provides a Cucumis melo var. inodorus plant regenerated from a tissue culture that comprises a hybrid genetic complement of this invention.

In still yet another aspect, the present invention provides a method of producing a plant derived from the Cucumis melo var. inodorus plants of the invention, the method comprising the steps of: (a) preparing a progeny plant derived from Cucumis melo var. inodorus plants of the invention, wherein said preparing comprises crossing a Cucumis melo var. inodorus plant of the invention with a second plant; and (b) crossing the progeny plant with itself or a second plant to produce a seed of a progeny plant of a subsequent generation. In further embodiments, the method may additionally comprise: (c) growing a progeny plant of a subsequent generation from said seed of a progeny plant of a subsequent generation and crossing the progeny plant of a subsequent generation with itself or a second plant; and repeating the steps for an additional 3-10 generations to produce a plant derived from Cucumis melo var. inodorus plants of the invention. The plant derived from Cucumis melo var. inodorus plants of the invention may be an inbred line, and the aforementioned repeated crossing steps may be defined as comprising sufficient inbreeding to produce the inbred line. In the method, it may be desirable to select particular plants resulting from step (c) for continued crossing according to steps (b) and (c). By selecting plants having one or more desirable traits, a plant derived from Cucumis melo var. inodorus plants of the invention is obtained which possesses some of the desirable traits of the line as well as potentially other selected traits.

In further aspects, the invention provides methods of determining the genotype of a Cucumis melo var. inodorus plant of the invention comprising obtaining a sample of nucleic acids from the plant and detecting in said nucleic acids a plurality of polymorphisms. Such methods may further comprise the step of storing the results of detecting the plurality of polymorphisms on a computer readable medium and the computer readable medium produced. Another aspect of the invention involves determining the genotype of a Cucumis melo var. inodorus plant of the invention comprising obtaining a sample of nucleic acids from the plant and determining the nucleic acid sequence of at least one locus.

In one aspect, the present invention provides a melon plant of the melon line HDG39-2022AN. Also provided are melon plants having all the physiological and morphological characteristics of such a plant. Parts of these melon plants are also provided, for example, including pollen, an ovule, scion, a rootstock, a fruit, and a cell of the plant.

Any embodiment discussed herein with respect to one aspect of the invention applies to other aspects of the invention as well, unless specifically noted.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

Other objects, features and advantages of the present invention will become apparent from the following detailed description. It should be understood, however, that the detailed description and any specific examples provided, while indicating specific embodiments of the invention, are given by way of illustration only, since various changes and modifications within the spirit and scope of the invention will become apparent to those skilled in the art from this detailed description.

### DETAILED DESCRIPTION OF THE FIGURES

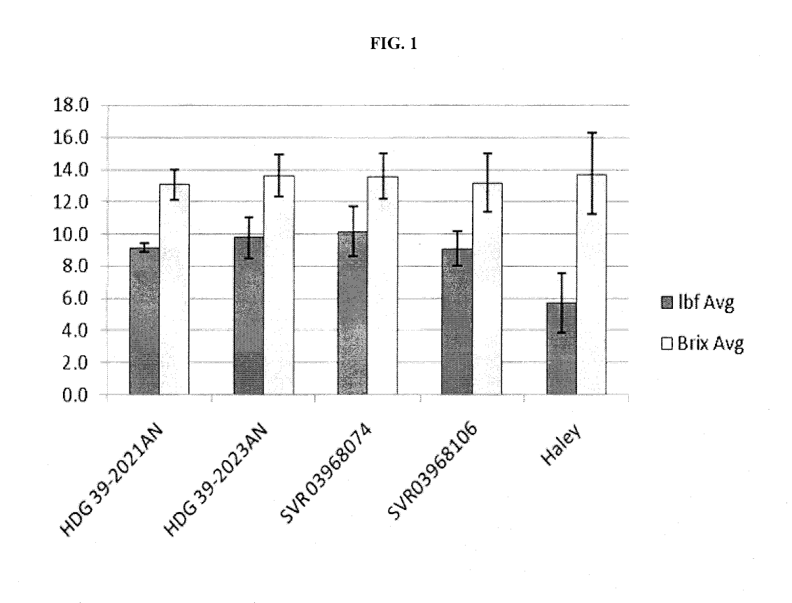

FIG. 1. Averages for flesh firmness and Brix of plants grown in Woodland, Calif., in the winter of year 21. Error bars represent standard deviation.

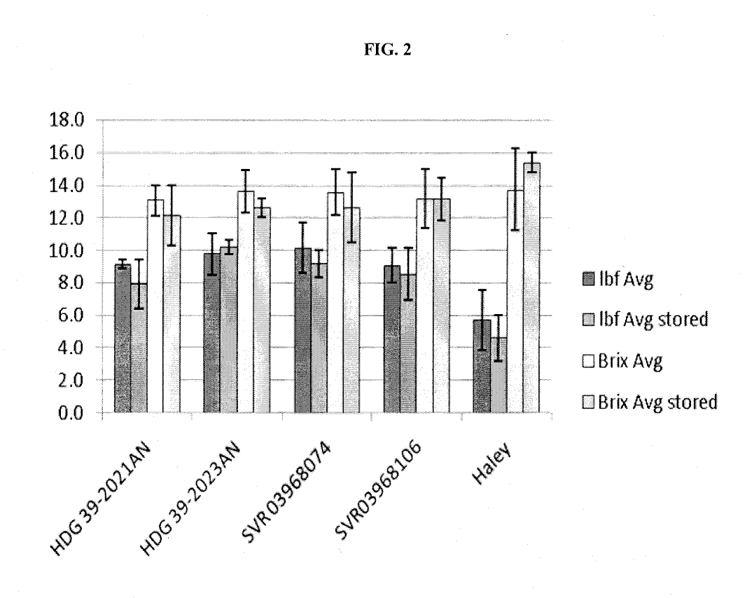

FIG. 2. Averages for flesh firmness and Brix, showing comparison of fresh measured melon fruit to fruit measured after cold storage for plants grown in Woodland, Calif. in the winter of year 21. Error bars represent standard deviation.

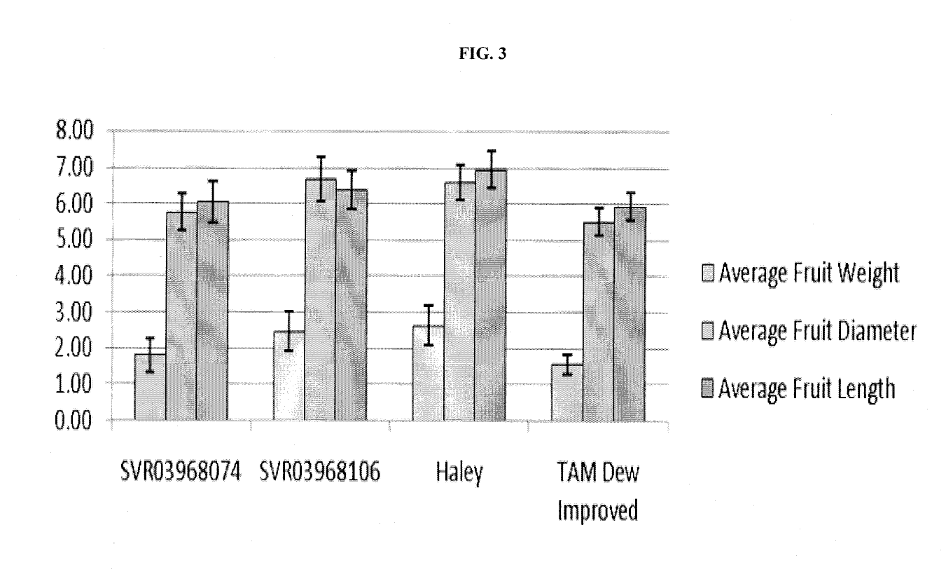

FIG. 3. Hybrid and selected variety comparison of averaged measurements for fruit weight, fruit diameter, and fruit length of plants grown in Chile in the spring of year 22. Error bars represent standard deviation.

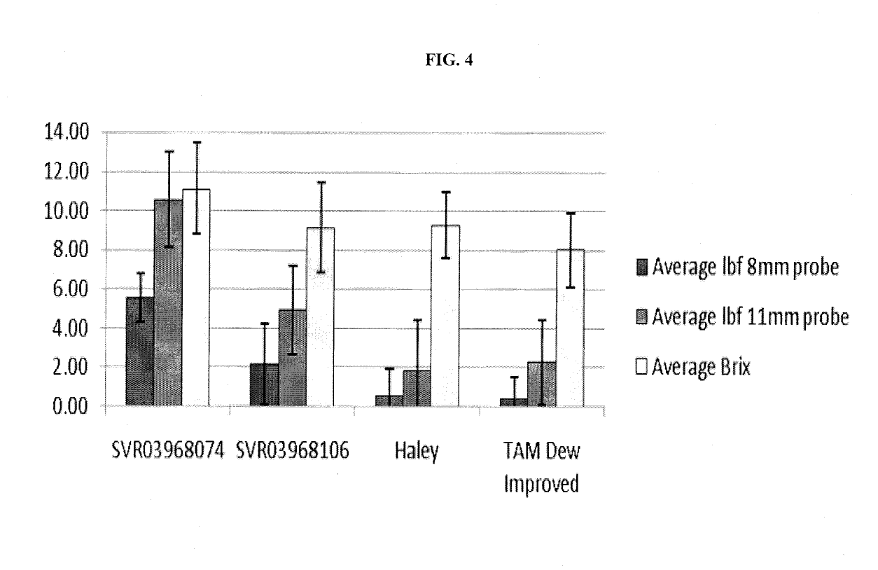

FIG. 4. Hybrid and selected variety comparison of averaged measurements for flesh firmness and Brix for plants grown in Chile in the spring of year 22. Error bars represent standard deviation.

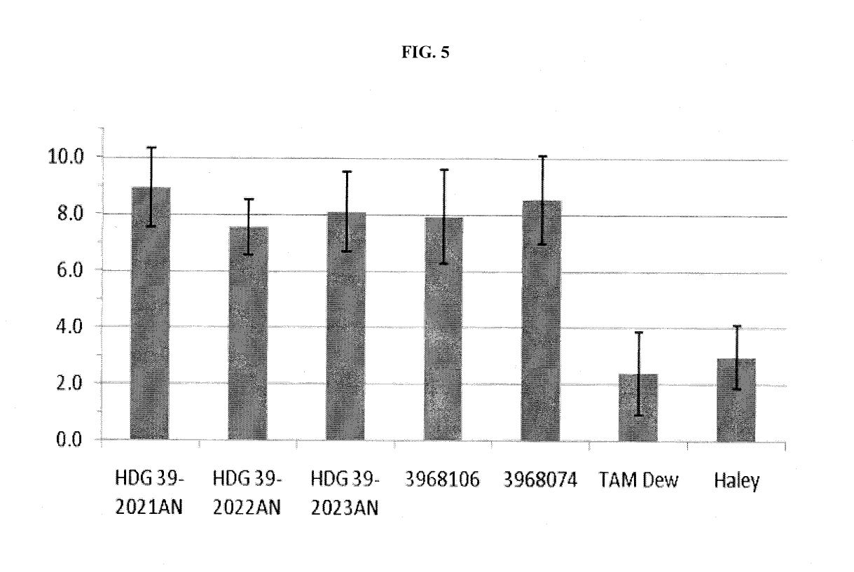

FIG. 5. Parent, hybrid and selected variety comparison of averaged measurements for flesh firmness taken with an 8 mm penetrometer tip for plants grown in Woodland, Calif. in the summer of year 22. Error bars represent standard deviation.

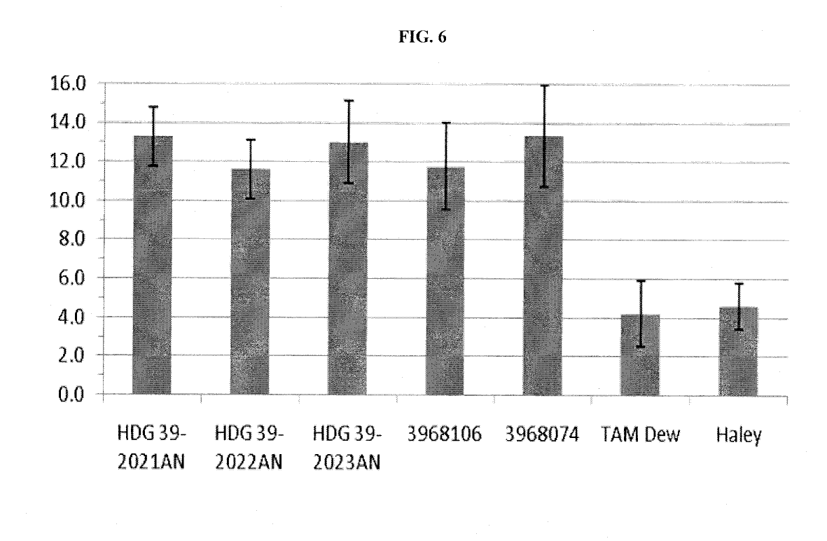

FIG. 6. Parent, hybrid and selected variety comparison of averaged measurements for flesh firmness taken with an 11 mm penetrometer tip for plants grown in Woodland, Calif. in the summer of year 22. Error bars represent standard deviation.

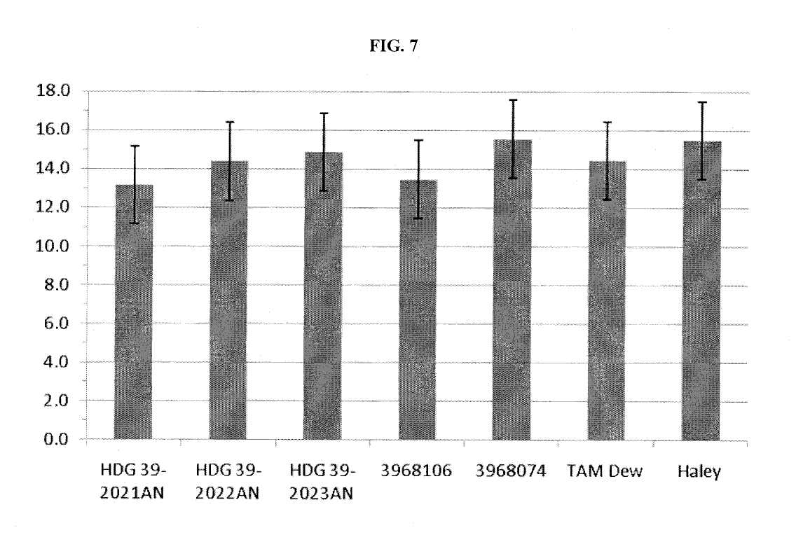

FIG. 7. Parent, hybrid and selected variety comparison of averaged measurements for Brix for plants grown in Woodland, Calif. in the summer of year 22. Error bars represent standard deviation.

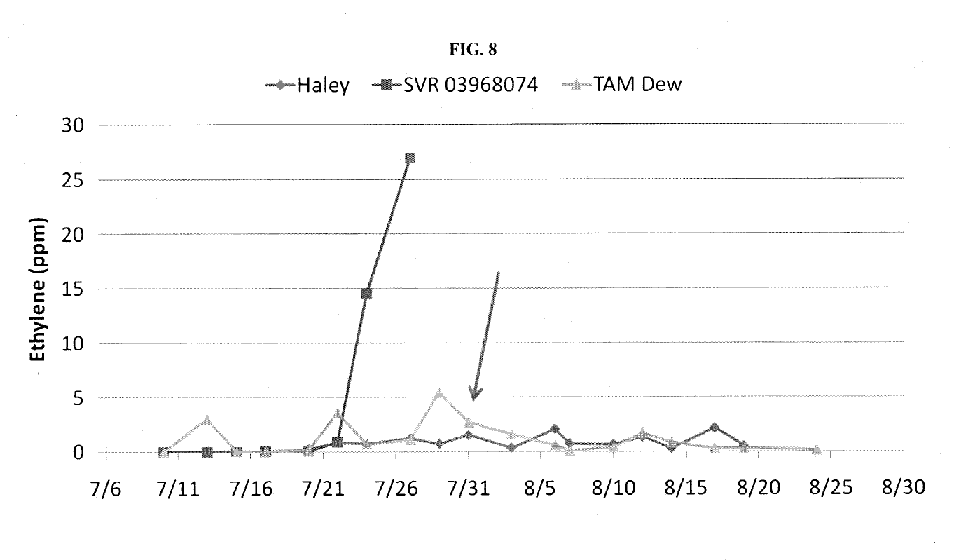

FIG. 8. Ethylene levels in fruits of SVR03968074, Haley, and TAM Dew over time, beginning from 31 days post pollination through abscission (for SVR03968074) or until the plants died (for Haley and TAM Dew). The approximate point of commercial harvest is indicated with the arrow. Each point on the graph is an average of three fruit.

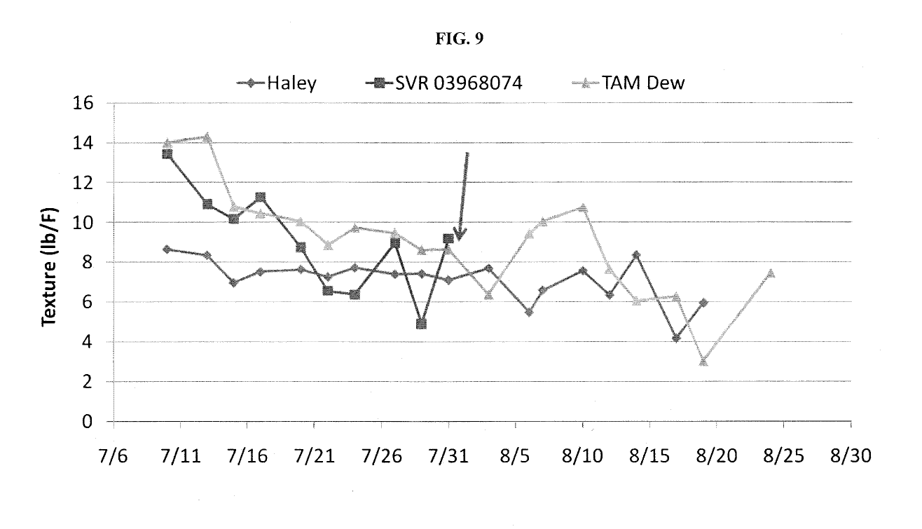

FIG. 9. Flesh firmness of fruits of SVR03968074, Haley, and TAM Dew over time, beginning from 31 days post pollination through abscission (for SVR03968074) or until the plants died (for Haley and TAM Dew). The approximate point of commercial harvest is indicated with the arrow. Each point on the graph is an average of three fruit.

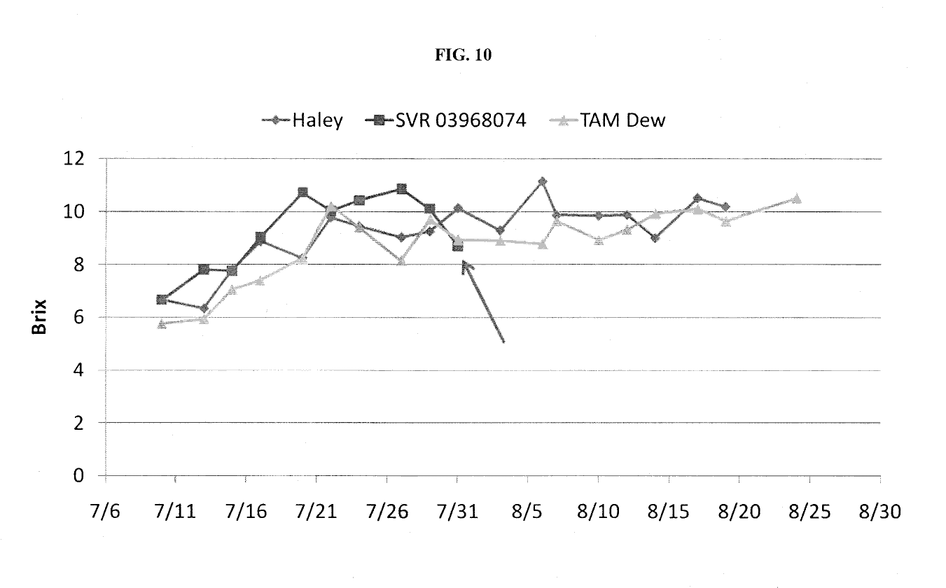

FIG. 10. Brix levels of fruits harvested from SVR03968074, Haley, and TAM Dew over time, beginning from 31 days post pollination through abscission (for SVR03968074) or until the plants died (for Haley and TAM Dew). The approximate point of commercial harvest is indicated with the arrow. Each point on the graph is an average of three fruit.

### DETAILED DESCRIPTION OF THE INVENTION

The present invention provides Cucumis melo var. inodorus melon plants of a commercially acceptable variety, and fruit thereof, with a harvest indicator that allows consistent assessment of fruit maturity at preferred harvest time. As used herein, the term “plant” includes plant cells, plant protoplasts, plant cells of tissue culture from which Cucumis melo var. inodorus melon plants can be regenerated, plant calli, plant clumps and plant cells that are intact in plants or parts of plants such as pollen, flowers, seed, leaves, stems and the like. Such Cucumis melo var. inodorus plants include plants of the market classes Piel de Sapo, Juan Canary (also known as and specifically including Jaune des Canaries and Amarillo), Earl's Type, Honeydew, Orange flesh honeydew, Hami Melon, Crenshaw and Casaba.

In one aspect, the invention provides a method of indication that melons have reached harvest maturity. The method provided for by this invention comprises harvest of a Cucumis melo var. inodorus melon exhibiting the slip trait, followed by immediate sale and consumption, or transport to market, followed by sale and consumption, or transport to a processing plant, followed by transport to market as a fresh cut product, followed by consumption. The invention detailed here is a slip harvest indicator in Cucumis melo var. inodorus melon lines. This invention provides for the slip trait to be used as an indicator of melon maturity. Upon exhibition of slip, the melon will be mature with firm flesh and may be consumed immediately or stored or transported for several weeks and still present a mature melon with flesh at a marketable firmness for whole or fresh cut markets.

In one aspect, the invention provides a visible and consistent measure of maturity on a per melon basis. This invention provides the exhibition of a trait and a method for determining the maturity of a melon. From this invention, the presence and degree of slip can now be utilized as an indicator of the maturity of a melon. The slip harvest indicator can be evaluated for each melon as a non-destructive indication of maturity. That is, depending on the presence or absence of slip observed for a melon, the melon can be harvested or left on the vine to reach a harvestable maturity.

The invention of a Cucumis melo var. inodorus slip harvest indicator also eases melon harvest, making the removal of the melon from the stem require less mechanical force. Exhibition of the slip trait is a separation of fruit from the stem or vine. Therefore, the slip process on a Cucumis melo var. inodorus melon fruit is the process by which the melon separates from the stem or vine. At full slip, the fruit may be completely separated from the vine, have a crack encircling the stem attachment with some attachment remaining in the center of the stem, or be almost separated with a high degree of slip where only a minimal force is needed to bring about full separation of the fruit from the vine.

In another aspect, the invention detailed here reduces wounding damage upon separation of the fruit from the vine. Current honeydew melon production harvest techniques involve cutting the melon from the stem. This practice results in a fruit with a portion of the stem attached where the wet cut end of the stem is now exposed to the environment. In the practice of this invention, the wound left at the point of melon connection to the vine has dried and sealed during the slip process. In this aspect, the harvest slip indicator provides a dried and sealed wound on a harvest mature fruit; a dried and sealed wound is significantly less prone to decay or infection than a wet or open wound or a wet cut stem end.

### A. DEFINITIONS

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses progeny back to one of the parents of the progeny to introduce one or more locus from one genetic background into another.

Commercially Acceptable Variety: Commercially acceptable traits vary according to market class and demand; for example, commercially acceptable honeydew melons, of the classification Cucumis melo var. inodorus have smooth skin with minimal or no presence of netting. All honeydew melon varieties may develop netting on the skin given precipitative environmental conditions, but a substantially smooth melon fits commercial preference. A commercially acceptable honeydew melon may be further defined by sugar content and flesh and rind color.

Crossing: The mating of two parent plants.

Cross-pollination: Fertilization by the union of two gametes from different plants.

Diploid: A cell or organism having two sets of chromosomes.

Emasculate: The removal of plant male sex organs or the inactivation of the organs with a cytoplasmic, nuclear genetic factor or a chemical agent conferring male sterility.

Enzymes: Molecules which can act as catalysts in biological reactions.

F₁ Hybrid: The first generation progeny of the cross of two nonisogenic plants.

Genotype: The genetic constitution of a cell or organism.

Haploid: A cell or organism having one set of chromosomes.

Harvest maturity: The stage at which a melon fruit is ready for harvest. In one embodiment, harvest maturity is marked by consumer-desirable and government-guided sugar levels. In certain embodiments and according to the California Code of Regulations for Honeydew Melons, the juice of the edible portion of the melon should not contain less than 10 percent soluble solids (10 degrees Brix).

Hybrid: An offspring of a cross between two genetically unlike individuals.

Inbred: A substantially homozygous individual or variety.

Introgress: Introduction of a new trait or genetic material from one plant or variety into another.

Linkage: A phenomenon wherein alleles on the same chromosome tend to segregate together more often than expected by chance if their transmission was independent.

Marker: A readily detectable genotype or phenotype, preferably inherited in codominant fashion (both alleles at a locus in a diploid heterozygote are readily detectable), with no environmental variance component, i.e., heritability of 1.

Maturity: The maturity of fruit development and optimal time for harvest. In one embodiment, the United States Department of Agriculture defines a “mature” honeydew as a melon that has reached the stage of maturity which will insure the proper completion of the normal ripening process (USDA. United States Standards for Grades of Honey Dew and Honey Ball Type Melons. 1997). In particular embodiments, fruit should be harvested at or substantially near maximum sweetness and flavor intensity.

Penetrometer: A device designed to measure force and used herein to measure fruit firmness. The device provides a quick, easy and accurate method to determine fruit flesh firmness. The data reported herein was gathered using a hand-held penetrometer to obtain pressure readings on mature fruit flesh. Specifically, Penetrometer model FDK30 (Force Dial FDK30 Wagner Instruments) was used to gather data. The unit of measure read by Penetrometer model FDK30 is “Pounds force”, or “lbf”, and is used herein to indicate readings made using a 8 millimeter ( 5/16 inch) or 11 millimeter ( 7/16 inch) probe, as indicated.

Phenotype: The detectable characteristics of a cell or organism, which characteristics are the manifestation of gene expression.

Quantitative Trait Loci (QTL): Quantitative trait loci (QTL) refer to genetic loci that control to some degree numerically representable traits that are usually continuously distributed.

Regeneration: The development of a plant from tissue culture.

Resistance: As used herein, the terms “resistance” and “tolerance” are used interchangeably to describe plants that show no symptoms to a specified biotic pest, pathogen, abiotic influence or environmental condition. These terms are also used to describe plants showing some symptoms but that are still able to produce marketable product with an acceptable yield. Some plants that are referred to as resistant or tolerant are only so in the sense that they may still produce a crop, even though the plants are stunted and the yield is reduced. Plants may be rated as exhibiting high resistance or intermediate resistance in order to distinguish differing levels of resistance.

Royal Horticultural Society (RHS) color chart value: The RHS color chart is a standardized reference which allows accurate identification of any color. A color's designation on the chart describes its hue, brightness and saturation. A color is precisely named by the RHS color chart by identifying the group name, sheet number and letter, e.g., Yellow-Orange Group 19A or Red Group 41B.

Self-pollination: The transfer of pollen from the anther to the stigma of the same plant.

Single Locus Converted (Conversion) Plant: Plants which are developed by a plant breeding technique called backcrossing, wherein essentially all of the desired morphological and physiological characteristics of a Cucumis melo var. inodorus variety are recovered in addition to the characteristics of the single locus transferred into the variety via the backcrossing technique and/or by genetic transformation.

Slip: Separation of melon fruit from plant stem. In some embodiments, slip is represented as quarter (¼), half (½), three-quarter (¾) or full slip where quarter slip represents 0-25% fruit detachment from the stem, half slip represents 26-50% fruit detachment from the stem, three-quarter slip represents 51-75% fruit detachment from the stem, and full slip represents 76-100% fruit detachment from the stem. As the stem attachment is roughly circular, this can be represented as 90, 180, 270, and 360 degrees of the circle being separated for quarter, half, three-quarter and full slip, respectively. In one embodiment, slip begins at 1 location on the circular fruit-stem attachment and spreads from there around the attachment. Half slip and greater may also be characterized by a slight depression of the stem end. At full slip, the stem may be more easily separated from the fruit. During the progression from start of slip until full slip, the fruit may be separated from the stem with increasingly less force. Slip may be marked by a visible abscission layer at the point of the stem's attachment to the fruit.

Soluble Solids: The percent of solid material found in edible fruit. As used herein, soluble solids are measured quantitatively with a refractometer as degrees Brix. Refractometers often include a sucrose scale, as Brix is formally defined as weight percent sucrose. If the only soluble solid present in an aqueous solution is sucrose, the sucrose scale should give the actual percentage sucrose. However, if other soluble solids are present, as is almost always the case, the reading is not equal to the percentage sucrose, but approximates the overall percentage of soluble solids in the sample. In short, although Brix is technically defined as weight percent sucrose, those of skill in the art recognize that weight percent soluble solids, as obtained with a refractometer, approximates weight percent sucrose and accurately indicates sweetness. Therefore, the higher the percentage soluble solids, as indicated by Brix, the higher the perceived sweetness of the fruit. Specifically, a Refractometer Atago PAL-1 (Atago 3810-PAL-1/VWR 47752-918) was used as described herein to measure Brix and report sweetness as degree Brix. Sweetness of a melon, may be measured quantitatively, as described above, using a refractometer, or qualitatively, by taste.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a Cucumis melo var. inodorus plant by transformation.

Variety: A group of similar plants that, by their genetic pedigrees and performance, can be identified from other varieties within the same species.

### B. CHARACTERISTICS OF CUCUMIS MELO VAR. INODORUS MELONS, PLANTS, AND PRODUCTION THEREOF

Honeydew melons, an example from the Cucumis melo var. inodorus group, have a substantially smooth, non-sutured, light green to cream-colored or light yellow to yellow skin with green, greenish white, or orange flesh. Young fruit may have some soft hairs present on the surface of the fruit. Commercially acceptable melons have smooth skin without the presence of netting. All honeydew melon varieties may develop netting on the skin given precipitative environmental conditions.

Sugar accumulation in the fruit is ongoing as melons go through the maturation process. Sugar levels in melon fruit do not increase after the fruit has been separated from the vine. The melon fruit maturation process is also marked by a steady transition of flesh firmness from hard to soft. That is, when melons are picked too early, the Brix levels are too low sugar is a major contributor to melon flavor and sugar deposition in the flesh does not continue after the fruit has been separated from the vine. When fruit are picked too late, the flesh is watery and becoming soft.

Cucumis melo var. inodorus are typically manually harvested by fruit maturity, the distinction of which is difficult to assess. No clear or consistent outer morphological characteristic is generally available aiding current honeydew melon maturity assessment. For example, no clear slip or fruit separation from the vine occurs in currently available honeydew melon varieties. In the absence of fruit separation from the vine, honeydews must be clipped from the plant at harvest. Fruit maturity and readiness for harvest may be determined by such factors as skin color, smoothness and fruit size. Although a larger melon may indicate a more mature fruit, melon size may differ depending on such factors as environmental conditions, plant health and number of fruit born per plant. Fruit color change and degree of smoothness may be subtle and/or differ between honeydew melon varieties, potentially necessitating a high degree of skill amongst harvesters.

Additionally, to determine approximate maturity of an entire field, one can randomly select melons and measure Brix levels with a portable refractometer device. This measurement requires access to a melon juice sample taken from within the melon and is a destructive measurement for the fruit that are sampled. This measurement will also be on a per field basis, still providing for potential harvest of immature individual melons.

Harvesting of melons may be followed by immediate consumption or by a period of transport to market. To address both of these circumstances, a melon should have significant sugar levels at the time of harvest to be determined “mature”, but should also retain flesh firmness through a window of time relevant to a period of transport to present high consumption quality when either immediately consumed and or when consumed post-transport.

### C. CHARACTERISTICS OF THE PRESENT INVENTION

Cucumis melo var. inodorus melon plants with separation from the stem, or slip, at harvest maturity are detailed here. This document provides details and methods for making and utilizing Cucumis melo var. inodorus melon lines that separate from the plant vine at harvest maturity. The lines of the invention detailed here exhibit flesh firmness at harvest and after a period of cold storage simulation of transport conditions.

Presently available commercially acceptable Cucumis melo var. inodorus melon varieties do eventually separate, or slip, from the vine; when they do, they are past prime consumption quality, with soft, watery flesh. Melon fruit flesh firmness is a desirable consumer trait in a harvested melon at the time of consumption. If no clear harvest indicator is available in current melon varieties, melon fruit may, under the relative skill of any given harvester at determining maturity, range from having low sugar levels and firm flesh to having high sugars levels and soft, watery flesh. The presently described innovation combines a means of determining melon maturity at an appropriate harvest time on a per melon basis, providing the means for consistent harvest of mature melons with high sugar levels and firm flesh.

Current commercial practice involves cutting Cucumis melo var. inodorus melons from the stem or vine at the time of harvest. This practice results in part of the stem remaining attached to the melon and creates, at the cut end of the stem, a wet wound that may be more prone to pathogen invasion and development. During the process of stem separation from the fruit, or slip, the wound dries and seals itself. Use of fruit separation from the fruit in lieu of cutting at harvest reduces the potential for fruit damage from pathogen development and invasion.

Use of fruit separation from the vine to assess harvest maturity additionally increases the speed with which melons may be harvested. Akin to the technique used for many other types of fruit and vegetables, for example tomatoes, assessment and subsequent harvest can be quickly assessed and asserted via application of force to assess the minimum of force necessary to remove the harvestable mature fruit from the stem.

Using standard crossing techniques, those of skill in the art may obtain melon fruit and plants of the present invention with desirable traits besides those described here, as the harvest maturity indicator may be inherited. For purposes of example and not mutual exclusivity, breeders may combine the fruit separation from vine harvest maturity indicator with a particular disease resistance.

### D. ORIGIN AND METHODS OF BREEDING LINES WITH A SLIP HARVEST INDICATOR

Development of slip, or separation from the vine, at harvest maturity in honeydew melon fruit has been demonstrated here in two ways, the first method utilized a unique concert of flesh firmness derived from a honeydew breeding population and introgression of slip from a melon type that separates from the vine at harvest maturity. The introgression of slip originated from a wide cross between a PI (Plant Introduction) line from the U.S. National Plant Germplasm System, PI 414723, and a commercial variety, Silver World (Known-You Seed Co., Taiwan). The second method utilizes a flesh firmness phenotype at the time of fruit separation from the vine to provide for a slip harvest indicator. See Example 1 for further information regarding line development. In accordance with this method, the trait may be introgressed, such as by backcrossing, into other Cucumis melo var. inodorus varieties by the methods provided herein.

### E. BREEDING PLANTS OF THE INVENTION

One aspect of the current invention concerns methods for crossing a plant of the invention with itself or a second plant and the seeds and plants produced by such methods. These methods can be used for propagation of plants of the invention or can be used to produce hybrid seeds and the plants grown therefrom. The seeds can be used by farmers in the commercial production of melons.

The plants of the present invention can be used for the development of new Cucumis melo var. inodorus plants involving introgression of one or more trait(s) of interest from a staring line, such as the slipping trait, into another genetic background. In selecting a second plant to cross with a plant of the invention for the purpose of developing novel Cucumis melo var. inodorus varieties, it will typically be preferred to choose those plants which either themselves exhibit one or more selected desirable characteristics or which exhibit the desired characteristic(s) alone or in hybrid combination. Examples of desirable characteristics include slipping, seed yield, germination, fruit size, fruit shape, rind coloring/striping, color of fruit flesh, seedling vigor, maturity, fruit yield, ease of fruit setting, fruit firmness, degrees of Brix, disease tolerance and adaptability for soil and climate conditions. Without limiting the scope of the disclosure, common inodorus melon market classes which could be used in breeding with plants of the present invention include: Piel de Sapo; Juan Canary (which is also known as Jaune des Canaries and Amarillo); Earl's Type; Honeydew; Orange flesh honeydew; Hami Melon; Crenshaw; and Casaba.

The plants of the present invention can beneficially be used as a male or female parent in the development of hybrid progeny. By selection of appropriate parents based on fruit shape (for example, round, blocky or long) and/or fruit size (for example, small, medium or large) progeny plants may be produced having fruit of the desired shape and size.

Cucumis melo var. inodorus plants of the invention can be crossed with a second Cucumis melo var. inodorus plant to produce first generation (F₁) progeny. The hybrid progeny are produced regardless of characteristics of the two varieties produced. As such, an F₁ hybrid honeydew plant may be produced by crossing plants of the invention with any second plant. The second plant may be genetically homogeneous (e.g., inbred) or may itself be a hybrid. Therefore, any F₁ hybrid plant produced by crossing plants according to the invention with a second plant is a part of the present invention.

In one embodiment, the present invention also provides plants according to the invention modified to include at least a first desired heritable trait. Such plants may, in one embodiment, be developed by a plant breeding technique called backcrossing, wherein essentially all of the desired morphological and physiological characteristics of a variety are recovered in addition to a genetic locus transferred into the plant via the backcrossing technique. The term single locus converted plant as used herein refers to those plants which are developed by a plant breeding technique called backcrossing, wherein essentially all of the desired morphological and physiological characteristics of a variety are recovered in addition to the single locus transferred into the variety via the backcrossing technique. Backcrossing methods can be used with the present invention to improve or introduce a characteristic into the present variety. The parental plant which contributes the locus for the desired characteristic is termed the nonrecurrent or donor parent. This terminology refers to the fact that the nonrecurrent parent is used one time in the backcross protocol and therefore does not recur. The parental plant to which the locus or loci from the nonrecurrent parent are transferred is known as the recurrent parent as it is used for several rounds in the backcrossing protocol.

In a typical backcross protocol, the original variety of interest (recurrent parent) is crossed to a second variety (nonrecurrent parent) that carries the single locus of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred locus from the nonrecurrent parent.

Uniform lines of new varieties may also be developed by way of double-haploids. This technique allows the creation of true breeding lines without the need for multiple generations of selfing and selection. In this manner, true breeding lines can be produced in as little as one generation. Haploid embryos may be produced from microspores, pollen, anther cultures, or ovary cultures. The haploid embryos may then be doubled autonomously, or by chemical treatments (e.g. colchicine treatment). Alternatively, haploid embryos may be grown into haploid plants and treated to induce chromosome doubling. In either case, fertile homozygous plants are obtained. In accordance with the invention, any of such techniques may be used in connection with plants according to the present invention and progeny thereof to achieve a homozygous line.

The selection of a suitable recurrent parent is an important step for a successful backcrossing procedure. The goal of a backcross protocol is to alter or substitute a single trait or characteristic in the original variety. To accomplish this, a single locus of the recurrent variety is modified or substituted with the desired locus from the nonrecurrent parent, while retaining essentially all of the rest of the desired genetic, and therefore the desired physiological and morphological constitution of the original variety. The choice of the particular nonrecurrent parent will depend on the purpose of the backcross; one of the major purposes is to add some commercially desirable trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Cucumis melo var. inodorus varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each.

Many single locus traits have been identified that are not regularly selected for in the development of a new inbred but that can be improved by backcrossing techniques. Single locus traits may or may not be transgenic; examples of these traits include, but are not limited to, herbicide resistance, resistance to bacterial, fungal, or viral disease, insect resistance, modified fatty acid or carbohydrate metabolism, and enhanced nutritional quality. These comprise genes generally inherited through the nucleus.

Direct selection may be applied where the single locus acts as a dominant trait. For such a selection process, the progeny of the initial cross and/or any subsequent cross can be selected to eliminate any plants which do not have the desired trait.

Selection of Cucumis melo var. inodorus plants for breeding is not necessarily dependent on the phenotype of a plant and instead can be based on genetic investigations. For example, one can utilize a suitable genetic marker which is closely genetically linked to a trait of interest. One of these markers can be used to identify the presence or absence of a trait in the offspring of a particular cross, and can be used in selection of progeny for continued breeding. This technique is commonly referred to as marker assisted selection. Any other type of genetic marker or other assay which is able to identify the relative presence or absence of a trait of interest in a plant can also be useful for breeding purposes. Procedures for marker assisted selection applicable to the breeding of melons are well known in the art. Such methods will be of particular utility in the case of recessive traits and variable phenotypes, or where conventional assays may be more expensive, time consuming or otherwise disadvantageous. Types of genetic markers which could be used in accordance with the invention include, but are not necessarily limited to, Simple Sequence Length Polymorphisms (SSLPs) (Williams et al., 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., 1998).

### F. PLANTS DERIVED FROM CUCUMIS MELO VAR. INODORUS PLANTS OF THE INVENTION BY GENETIC ENGINEERING

Many useful traits that can be introduced by backcrossing, as well as directly into a plant, are those which are introduced by genetic transformation techniques. Genetic transformation may therefore be used to insert a selected transgene into plants of the invention or may, alternatively, be used for the preparation of transgenes which can be introduced by backcrossing. Methods for the transformation of plants, including Cucumis melo var. inodorus plants, are well known to those of skill in the art. Techniques which may be employed for the genetic transformation of melons include, but are not limited to, electroporation, microprojectile bombardment, Agrobacterium-mediated transformation and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

A particularly efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target plant cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., 1985; U.S. Pat. No. 5,563,055).

Transformation of plant protoplasts also can be achieved using methods based on calcium phosphate precipitation, polyethylene glycol treatment, electroporation, and combinations of these treatments (see, e.g., Potrykus et al., 1985; Omirulleh et al., 1993; Fromm et al., 1986; Uchimiya et al., 1986; Marcotte et al., 1988). Transformation of Cucumis melo var. inodorus plants and expression of foreign genetic elements is exemplified in Choi et al. (1994) and Ellul et al. (2003).

A number of promoters have utility for plant gene expression for any gene of interest including but not limited to selectable markers, scoreable markers, genes for pest tolerance, disease resistance, nutritional enhancements and any other gene of agronomic interest. Examples of constitutive promoters useful for plant gene expression include, but are not limited to, the cauliflower mosaic virus (CaMV) P-35S promoter, which confers constitutive, high-level expression in most plant tissues (see, e.g., Odel et al., 1985), including monocots (see, e.g., Dekeyser et al., 1990; Terada and Shimamoto, 1990); a tandemly duplicated version of the CaMV 35S promoter, the enhanced 35S promoter (P-e35S) the nopaline synthase promoter (An et al., 1988), the octopine synthase promoter (Fromm et al., 1989); and the figwort mosaic virus (P-FMV) promoter as described in U.S. Pat. No. 5,378,619 and an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, and other plant DNA virus promoters known to express in plant cells.

A variety of plant gene promoters that are regulated in response to environmental, hormonal, chemical, and/or developmental signals can be used for expression of an operably linked gene in plant cells, including promoters regulated by (1) heat (Callis et al., 1988), (2) light (e.g., pea rbcS-3A promoter, Kuhlemeier et al., 1989; maize rbcS promoter, Schaffner and Sheen, 1991; or chlorophyll a/b-binding protein promoter, Simpson et al., 1985), (3) hormones, such as abscisic acid (Marcotte et al., 1989), (4) wounding (e.g., wunl, Siebertz et al., 1989); or (5) chemicals such as methyl jasmonate, salicylic acid, or Safener. It may also be advantageous to employ organ-specific promoters (e.g., Roshal et al., 1987; Schernthaner et al., 1988; Bustos et al.,) or constitutively-expressed plant promoters.

Exemplary nucleic acids which may be introduced to the plants of this invention include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into a plant according to the invention. Non-limiting examples of particular genes and corresponding phenotypes one may choose to introduce into a plant include one or more genes for insect tolerance, such as a Bacillus thuringiensis (B.t.) gene, pest tolerance such as genes for fungal disease control, herbicide tolerance such as genes conferring glyphosate tolerance, and genes for quality improvements such as yield, nutritional enhancements, environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

Alternatively, the DNA coding sequences can affect these phenotypes by encoding a non-translatable RNA molecule that causes the targeted inhibition of expression of an endogenous gene, for example via antisense- or cosuppression-mediated mechanisms (see, for example, Bird et al., 1991). The RNA could also be a catalytic RNA molecule (i.e., a ribozyme) engineered to cleave a desired endogenous mRNA product (see for example, Gibson and Shillito, 1997). Thus, any gene which produces a protein or mRNA which expresses a phenotype or morphology change of interest is useful for the practice of the present invention.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Generation of Inbred Lines

The original cross in the development of HDG39-2021AN was between two Seminis inbred breeding lines. Parent 1 was BC₃S₆ (PI 414723×Silver World) (PI 414723 (USDA), Silver World (Known-You Seed Co., Taiwan)) and Parent 2 was F₇ (TAM Dew×Silver World) (TAM Dew (Texas A & M University, Texas U.S.A.), Silver World (Known-You Seed Co., Taiwan)). Table 1 details subsequent generations and selections for a harvest maturity indicator of slipping stem attachment, or fruit separation from stem at harvest maturity, and flesh firmness.

TABLE 1
HDG39-2021AN (female of SVR03968106 hybrid) pedigree and trait selections

| Year | Line ID | | Selection |
|---|---|---|---|
| | | BC₃S₆(PI414723 × Silver World) | Inbred breeding line |
| | | F₇(TAM Dew × Silver World) | Inbred breeding line |
| 10 | | BC₃S₆(PI414723 × Silver World) × | |
| | | F₇(TAM Dew × Silver World) | |
| | | [same as: BC₃S₆(6577151PMR) × | |
| | | F₇(TAM151)] | |
| 11 Spring | | F₂(BC₃S₆(PI414723 × Silver | slipping stem attachment, |
| | | World) × F₇(TAM Dew × Silver | firm flesh |
| | | World)) | |
| 11 Fall | | F₃(BC₃S₆(PI414723 × Silver | all selections had slipping |
| | | World) × F₇(TAM Dew × Silver | stem attachment, firm flesh |
| | | World)) | |
| 12 Spring | | F₄(BC₃S₆(PI414723 × Silver | firm flesh |
| | | World) × F₇(TAM Dew × Silver | |
| | | World)) | |
| 12 Fall | | F₅(BC₃S₆(PI414723 × Silver | firm flesh |
| | | World) × F₇(TAM Dew × Silver | |
| | | World)) | |
| 13 Spring | | F₆(BC₃S₆(PI414723 × Silver | slipping stem attachment, |
| | | World) × F₇(TAM Dew × Silver | |
| | | World)) | |
| 13 Fall | | F₇(BC₃S₆(PI414723 × Silver | firm flesh |
| | | World) × F₇(TAM Dew × Silver | |
| | | World)) | |
| 14 Spring | | F₈(BC₃S₆(PI414723 × Silver | firm flesh |
| | | World) × F₇(TAM Dew × Silver | |
| | | World)) | |
| 14 Fall | | F₉(BC₃S₆(PI414723 × Silver | firm flesh |
| | | World) × F₇(TAM Dew × Silver | |
| | | World)) | |
| 17 Spring | HDG39- | F₁₀(BC₃S₆(PI414723 × Silver | No selection - seed |
| | 2021AN | World) × F₇(TAM Dew × Silver | increase |
| | | World)) | |

Table 2 details the generation and selections for the inbred line HDG39-2023AN.

TABLE 2
HDG39-2023AN (male of SVR03968106 and SVR03968074 hybrids) pedigree
and trait selections

| Year | Line ID | | Selection |
|---|---|---|---|
| | | F₈(TAM Dew × Silver World) | Inbred breeding line |
| | | F₅(Silver World) | Inbred breeding line |
| 11 | | F₁ SVR03912988 = F₁((F₈(TAM | |
| | | Dew × Silver World)) × (F₅(Silver | |
| | | World)) | |
| 15 Spring | | F₂((F₈(TAM Dew × Silver World)) × | firm flesh |
| | | F₅(Silver World)) | |
| 16 Spring | | F₃((F₈(TAM Dew × Silver World)) × | firm flesh, slipping stem |
| | | F₅(Silver World)) | attachment |
| 16 Fall | | F₄((F₈(TAM Dew × Silver World)) × | firm flesh |
| | | F₅(Silver World)) | |
| 17 Spring | | F₅((F₈(TAM Dew × Silver World)) × | firm flesh, slipping stem |
| | | F₅(Silver World)) | attachment |
| 18 Fall | | F₆((F₈(TAM Dew × Silver World)) × | firm flesh |
| | | F₅(Silver World)) | |
| 19 Spring | | F₇((F₈(TAM Dew × Silver World)) × | firm flesh |
| | | F₅(Silver World)) designated | |
| | | HDG39-2023AN | |
| 21 | HDG39- | F₈((F₈(TAM Dew × Silver World)) × | No selection - seed |
| Summer | 2023AN | F₅(Silver World)) | increase: all fruits with |
| | | | slipping stem attachment |

Table 3 describes the generation and selections for the inbred line HDG29-2022AN.

TABLE 3
HDG39-2022AN (female of SVR03968074 hybrid) pedigree and
trait selections

| Year | Line ID | Generation | Selection |
|---|---|---|---|
| 1 | F1(TAM151 × HP279) | F1 | |
| 2 | 01F 1622-6 OP | F2 | Firm flesh, high humidity adaptation |
| 3 | 03Tx 2065-1 | F3 | Plant health, fruit size, fruit shape, firm |
| | | | flesh, Brix |
| 3 | 03GH 777-9 | F4 | Plant health, fruit size, fruit shape, firm |
| | | | flesh, Brix |
| 4 | 04GH 94-3 | F5 | Plant health, fruit size, fruit shape, firm |
| | | | flesh, Brix |
| 4 | 04GH 725-11 | F6 | Plant health, fruit size, fruit shape, firm |
| | | | flesh, Brix |
| 5 | 05GH 192-9 | F7 | Plant health, fruit size, fruit shape, firm |
| | | | flesh, Brix |
| 6 | 06GH 234-M | F8 | Plant health, fruit size, fruit shape, firm |
| | | | flesh, Brix |
| 7 | 08GH 482-M | F9 | Breeder seed increase (named HDG39- |
| | | | 2022AN) |

### Example 2

### Generation of Hybrid Lines

Lines SVR03968074 and SVR03968106 are hybrid lines derived from two different approaches for development of a honeydew harvest maturity indicator: Honeydew×Honeydew and PI 414723×Honeydew crosses, respectively.

SVR03968106 is the hybrid of a HDG39-2021AN (female)×HDG39-2023AN (male) cross where HDG39-2023AN is a slipping honeydew from a Honeydew×Honeydew population and HDG39-2021AN is a slipping honeydew from a PI414723×Honeydew population (see Example 1 for pedigree information).

SVR03968074 is the hybrid of the HDG29-2022AN (female)×HDG39-2023AN (male) cross where HDG29-2022AN is a non-slipping line and HDG39-2023AN is a slipping line and both are from a Honeydew×Honeydew population (see Example 1 for pedigree information).

### Example 3

### Flesh Firmness at Slip and Comparison to Commercial Hybrids

When compared to commercial varieties late in the growing season in the Summer of year 21, hybrid varieties of the instant invention exhibit improved flesh firmness. Commercial standards represented here are TAM Dew (Texas A & M public release), a standard open pollinated (OP) variety, and Haley (Shamrock Seed), a commercial hybrid.

### Example 4

### Demonstration of Harvest Maturity and Flesh Firmness at Fruit Separation from Stem and Flesh Firmness After Transport Simulation Storage

Test measurements were carried out on melon fruit samples grown in Woodland, Calif. greenhouses in the winter of year 21 (Table 4). FIGS. 1 and 2 represent the averages from Table 4. For flesh firmness readings, melon fruit were cut in half and a penetrometer reading was taken on melon flesh halfway between the interior cavity edge and the fruit skin. For Brix readings, a fruit slice capturing all the space between the interior cavity and the fruit skin was cut and juice was squeezed directly into the refractometer for analysis.

TABLE 4
Comparison of measurements for flesh firmness and Brix for parent, hybrid
and selected lines grown in Woodland, CA in the winter of year 21

| | Measurement | Lbf | | | Storage |
|---|---|---|---|---|---|
| Line | Date | (11 mm tip) | Brix | Slip | duration |
| HDG39-2021AN | 2-Dec | 9.0 | 14.1 | Full Slip | |
| | 9-Dec | 9.5 | 12.8 | Full Slip | |
| | 9-Dec | 9.0 | 12.3 | Full Slip | |
| | 18-Dec | 8.0 | 11.7 | Full Slip | Storage 14 days |
| | 18-Dec | 6.0 | 10.8 | ½ Slip | Storage 14 days |
| | 18-Dec | 8.5 | 14.0 | Full Slip | Storage 14 days |
| | 18-Dec | 7.0 | 14.1 | Full Slip | Storage 14 days |
| | 18-Dec | 10.0 | 10.1 | Full Slip | Storage 14 days |
| HDG39-2023AN | 17-Nov | 7.0 | 14.7 | Full Slip | |
| | 17-Nov | 8.5 | 12.1 | Full Slip | |
| | 24-Nov | 10.0 | 12.9 | Full Slip | |
| | 24-Nov | 12.0 | 13.5 | Full Slip | |
| | 24-Nov | 11.0 | 11.5 | Full Slip | |
| | 24-Nov | 11.0 | 11.9 | Full Slip | |
| | 24-Nov | 11.0 | 14.0 | Full Slip | |
| | 25-Nov | 12.0 | 15.1 | Full Slip | |
| | 25-Nov | 10.0 | 13.5 | Full Slip | |
| | 25-Nov | 10.0 | 11.6 | Full Slip | |
| | 25-Nov | 9.0 | 15.4 | Full Slip | |
| | 25-Nov | 10.0 | 16.1 | Full Slip | |
| | 2-Dec | 8.0 | 13.0 | Full Slip | |
| | 2-Dec | 10.0 | 13.7 | Full Slip | |
| | 2-Dec | 9.0 | 14.7 | Full Slip | |
| | 2-Dec | 9.0 | 14.6 | Full Slip | |
| | 2-Dec | 9.0 | 13.1 | Full Slip | |
| | 2-Dec | 10.0 | 12.8 | Full Slip | |
| | 2-Dec | 10.0 | 14.9 | Full Slip | |
| | 2-Dec | 9.0 | 13.8 | Full Slip | |
| | 18-Dec | 10.0 | 12.1 | Full Slip | Storage 28 days |
| | 18-Dec | 10.0 | 11.9 | Full Slip | Storage 28 days |
| | 18-Dec | 11.0 | 12.8 | Full Slip | Storage 28 days |
| | 18-Dec | 10.0 | 13.0 | Full Slip | Storage 28 days |
| | 18-Dec | 10.0 | 13.2 | Full Slip | Storage 28 days |
| SVR03968074 | 17-Nov | 8.5 | 12.4 | Full Slip | |
| | 17-Nov | 8.0 | 9.7 | Full Slip | |
| | 24-Nov | 10.0 | 10.7 | Full Slip | |
| | 24-Nov | 12.0 | 13.8 | Full Slip | |
| | 24-Nov | 12.0 | 13.7 | Full Slip | |
| | 24-Nov | 12.5 | 12.4 | Full Slip | |
| | 24-Nov | 12.0 | 12.6 | Full Slip | |
| | 25-Nov | 8.0 | 13.0 | Full Slip | |
| | 25-Nov | 9.0 | 14.3 | Full Slip | |
| | 25-Nov | 9.0 | 13.2 | Full Slip | |
| | 2-Dec | 9.0 | 14.6 | Full Slip | |
| | 2-Dec | 10.0 | 13.8 | Full Slip | |
| | 2-Dec | 10.0 | 13.9 | Full Slip | |
| | 2-Dec | 9.0 | 14.9 | Full Slip | |
| | 2-Dec | 8.0 | 15.8 | Full Slip | |
| | 2-Dec | 9.0 | 14.8 | Full Slip | |
| | 2-Dec | 10.0 | 13.4 | Full Slip | |
| | 2-Dec | 10.0 | 13.6 | Full Slip | |
| | 2-Dec | 12.0 | 14.1 | Full Slip | |
| | 2-Dec | 12.0 | 14.5 | Full Slip | |
| | 2-Dec | 11.0 | 14.9 | Full Slip | |
| | 2-Dec | 12.0 | 14.4 | Full Slip | |
| | 18-Dec | 9.0 | 14.9 | Full Slip | Storage 28 days |
| | 18-Dec | 8.0 | 13.8 | Full Slip | Storage 28 days |
| | 18-Dec | 10.0 | 10.5 | Full Slip | Storage 28 days |
| | 18-Dec | 9.0 | 10.2 | Full Slip | Storage 28 days |
| | 18-Dec | 10.0 | 13.9 | Full Slip | Storage 28 days |
| SVR03968106 | 24-Nov | 9.0 | 13.9 | Full Slip | |
| | 24-Nov | 8.0 | 15.5 | Full Slip | |
| | 24-Nov | 7.0 | 8.8 | Full Slip | |
| | 24-Nov | 9.5 | 11.8 | Full Slip | |
| | 24-Nov | 10.0 | 13.0 | Full Slip | |
| | 24-Nov | 10.0 | 10.2 | Full Slip | |
| | 25-Nov | 8.0 | 13.1 | Full Slip | |
| | 25-Nov | 10.0 | 14.7 | Full Slip | |
| | 2-Dec | 9.0 | 12.6 | Full Slip | |
| | 2-Dec | 11.0 | 14.3 | Full Slip | |
| | 2-Dec | 9.0 | 10.2 | Full Slip | |
| | 2-Dec | 9.0 | 13.5 | Full Slip | |
| | 2-Dec | 9.0 | 12.8 | Full Slip | |
| | 2-Dec | 10.0 | 12.8 | Full Slip | |
| | 2-Dec | 10.0 | 13.0 | Full Slip | |
| | 2-Dec | 9.0 | 13.3 | Full Slip | |
| | 2-Dec | 9.0 | 14.9 | Full Slip | |
| | 2-Dec | 10.0 | 15.4 | Full Slip | |
| | 2-Dec | 8.0 | 14.7 | Full Slip | |
| | 2-Dec | 7.0 | 15.2 | Full Slip | |
| | 18-Dec | 9.0 | 15.3 | ½ Slip | Storage 7 days |
| | 18-Dec | 10.0 | 14.6 | Full Slip | Storage 7 days |
| | 18-Dec | 9.0 | 12.5 | Full Slip | Storage 7 days |
| | 18-Dec | 10.0 | 14.2 | Full Slip | Storage 7 days |
| | 18-Dec | 10.0 | 11.8 | Full Slip | Storage 7 days |
| | 18-Dec | 8.5 | 12.8 | Full Slip | Storage 28 days |
| | 18-Dec | 5.0 | 12.3 | Full Slip | Storage 28 days |
| | 18-Dec | 6.5 | 14.1 | Full Slip | Storage 28 days |
| | 18-Dec | 9.0 | 11.4 | Full Slip | Storage 28 days |
| | 18-Dec | 8.5 | 12.4 | Full Slip | Storage 28 days |
| Haley | 2-Dec | 4.5 | | Full Slip | |
| | 9-Dec | 5.0 | 16.4 | Full Slip | |
| | 9-Dec | 5.0 | 13.3 | Full Slip | |
| | 9-Dec | 4.5 | 16.1 | Full Slip | |
| | 15-Dec | 3.5 | 13.4 | Full Slip | |
| | 15-Dec | 10.0 | 9.3 | Full Slip | |
| | 15-Dec | 4.5 | 15.4 | Full Slip | |
| | 15-Dec | 8.0 | 15.1 | Full Slip | |
| | 15-Dec | 5.5 | 13.2 | Full Slip | |
| | 15-Dec | 5.5 | 14.5 | Full Slip | |
| | 15-Dec | 6.5 | 15.3 | Full Slip | |
| | 15-Dec | 5.0 | 9.0 | Full Slip | |
| | 18-Dec | 3.0 | 15.3 | Full Slip | Storage 14 days |
| | 18-Dec | 3.5 | 15.2 | Full Slip | Storage 14 days |
| | 18-Dec | 4.5 | 15.1 | Full Slip | Storage 14 days |
| | 18-Dec | 4.0 | 16.0 | Full Slip | Storage 14 days |
| | 18-Dec | 4.0 | 15.6 | Full Slip | Storage 7 days |
| | 18-Dec | 3.5 | 14.6 | Full Slip | Storage 7 days |
| | 18-Dec | 5.5 | 16.1 | Full Slip | Storage 7 days |
| | 18-Dec | 6.5 | 16.1 | Full Slip | Storage 7 days |
| | 18-Dec | 7.0 | 14.6 | Full Slip | Storage 7 days |

### Example 5

### Demonstration of Harvest Maturity and Flesh Firmness at Fruit Separation from Stem

Test measurements were carried out on melon fruit samples grown in the field in Chile in the spring of year 22 (Table 5). FIGS. 3 and 4 represent the averages from Table 5. For flesh firmness readings, melon fruit were cut in half and a penetrometer reading was taken on melon flesh halfway between the interior cavity edge and the fruit skin. For Brix readings, a fruit slice capturing all the space between the interior cavity and the fruit skin was cut and juice was squeezed directly into the refractometer for analysis.

TABLE 5
Comparison of measurements for fruit weight, fruit diameter, fruit length, flesh firmness
and Brix for hybrid and selected lines grown in Chile in the spring of year 22

| | | | | | | lbf | lbf | |
|---|---|---|---|---|---|---|---|---|
| Measurement | | Fruit | Fruit | Fruit | Fruit | 8 mm | 11 mm | |
| Date | Variety | Count | Weight | Diameter | Length | probe | probe | Brix |
| 24-Feb | SVR03968074 | 20 | 2.18 | 6.25 | 6.25 | 5.50 | 8.75 | 14.00 |
| | | | 1.48 | 5.13 | 5.50 | 9.00 | 17.00 | 8.10 |
| | | | 1.43 | 5.50 | 5.25 | 5.00 | 9.00 | 12.40 |
| | | | 1.73 | 5.50 | 6.25 | 5.00 | 11.75 | 12.20 |
| | | | 1.92 | 6.00 | 6.50 | 4.50 | 7.00 | 11.30 |
| | | | 1.43 | 5.75 | 5.25 | 5.75 | 11.25 | 9.90 |
| | | | 1.90 | 5.75 | 6.50 | 5.75 | 10.00 | 8.20 |
| | | | 1.83 | 5.88 | 5.88 | 4.25 | 8.75 | 12.60 |
| | | | 2.61 | 6.50 | 7.00 | 4.50 | 10.00 | 12.30 |
| | | | 2.00 | 5.75 | 6.50 | 6.75 | 10.50 | 12.00 |
| | | | 2.10 | 6.25 | 6.25 | 4.50 | 9.25 | 14.50 |
| | | | 1.50 | 5.50 | 5.50 | 6.50 | 12.50 | 11.30 |
| | | | 2.19 | 6.25 | 6.50 | 4.75 | 11.00 | 10.40 |
| | | | 2.53 | 6.50 | 6.50 | 7.75 | 14.00 | 12.20 |
| | | | 1.23 | 5.00 | 5.50 | 6.25 | 11.00 | 11.30 |
| | | | 2.50 | 6.50 | 6.75 | 4.50 | 9.00 | 13.90 |
| | | | 1.10 | 5.25 | 5.25 | 6.00 | 14.00 | 8.90 |
| | | | 1.60 | 5.50 | 5.75 | 4.50 | 9.25 | 12.50 |
| | | | 1.62 | 5.50 | 6.50 | 4.50 | 10.00 | 9.40 |
| | | | 1.12 | 5.00 | 5.50 | 5.25 | 7.25 | 5.10 |
| | | Avg. | 1.80 | 5.76 | 6.04 | 5.53 | 10.56 | 11.13 |
| | | St. Dev. | 0.46 | 0.49 | 0.56 | 1.25 | 2.41 | 2.31 |
| 24-Feb | SVR03968106 | 27 | 1.75 | 6.00 | 5.75 | 4.50 | 6.25 | 5.90 |
| | | | 2.20 | 6.50 | 6.75 | 3.25 | 4.25 | 6.20 |
| | | | 3.60 | 7.75 | 7.25 | 0.00 | 3.25 | 11.10 |
| | | | 1.93 | 6.25 | 5.75 | 0.00 | 5.25 | 6.20 |
| | | | 2.16 | 6.75 | 6.13 | 0.00 | 3.50 | 10.20 |
| | | | 2.90 | 7.00 | 6.75 | 3.25 | 3.75 | 7.40 |
| | | | 2.20 | 6.75 | 6.00 | 3.50 | 6.00 | 7.50 |
| | | | 2.50 | 6.50 | 6.50 | 3.25 | 3.75 | 9.40 |
| | | | 2.64 | 7.00 | 6.25 | 3.50 | 4.25 | 7.00 |
| | | | 2.30 | 6.50 | 5.88 | 0.00 | 0.00 | 9.20 |
| | | | 2.75 | 6.75 | 7.00 | 4.50 | 7.25 | 10.40 |
| | | | 3.50 | 7.50 | 7.25 | 5.00 | 8.00 | 13.50 |
| | | | 2.60 | 6.75 | 6.75 | 0.00 | 3.25 | 13.30 |
| | | | 2.40 | 6.75 | 6.50 | 5.25 | 9.00 | 9.90 |
| | | | 1.64 | 5.75 | 5.88 | 0.00 | 4.50 | 8.50 |
| | | | 2.80 | 6.88 | 7.00 | 4.00 | 7.50 | 10.70 |
| | | | 2.50 | 7.75 | 6.50 | 0.00 | 3.25 | 7.80 |
| | | | 2.93 | 7.25 | 6.75 | 3.25 | 5.75 | 10.10 |
| | | | 2.58 | 6.50 | 6.25 | 0.00 | 4.00 | 9.40 |
| | | | 1.90 | 6.00 | 6.00 | 3.50 | 5.00 | 9.30 |
| | | | 1.78 | 6.00 | 5.50 | 0.00 | 4.50 | 6.00 |
| | | | 2.24 | 6.75 | 6.00 | 3.25 | 7.00 | 6.80 |
| | | | 2.94 | 6.88 | 6.50 | 0.00 | 4.00 | 12.80 |
| | | | 2.00 | 6.14 | 6.00 | 0.00 | 5.25 | 11.00 |
| | | | 1.60 | 5.13 | 5.25 | 3.25 | 5.50 | 7.00 |
| | | | 2.90 | 7.25 | 6.88 | 0.00 | 0.00 | 8.30 |
| | | | 3.60 | 7.50 | 7.00 | 5.25 | 9.50 | 12.20 |
| | | Avg. | 2.48 | 6.69 | 6.37 | 2.17 | 4.94 | 9.15 |
| | | St. Dev. | 0.56 | 0.62 | 0.54 | 2.06 | 2.25 | 2.27 |
| 24-Feb | Haley | 29 | 3.07 | 6.75 | 7.25 | 0.00 | 0.00 | 8.50 |
| | | | 2.49 | 6.50 | 6.50 | 0.00 | 0.00 | 10.40 |
| | | | 2.40 | 6.50 | 6.50 | 0.00 | 0.00 | 11.50 |
| | | | 2.30 | 6.25 | 6.50 | 0.00 | 0.00 | 8.00 |
| | | | 1.92 | 5.75 | 6.50 | 0.00 | 4.25 | 8.00 |
| | | | 1.62 | 5.75 | 6.25 | 0.00 | 3.25 | 7.50 |
| | | | 2.60 | 6.63 | 7.00 | 3.50 | 5.50 | 8.70 |
| | | | 1.87 | 6.00 | 6.25 | 0.00 | 3.75 | 11.20 |
| | | | 1.72 | 5.50 | 6.50 | 4.75 | 7.50 | 5.10 |
| | | | 2.30 | 6.25 | 6.75 | 0.00 | 4.25 | 9.20 |
| | | | 3.00 | 7.00 | 6.75 | 0.00 | 0.00 | 10.90 |
| | | | 2.00 | 5.75 | 6.75 | 0.00 | 0.00 | 9.80 |
| | | | 2.10 | 6.25 | 6.75 | 0.00 | 3.50 | 8.60 |
| | | | 2.14 | 6.50 | 6.50 | 3.25 | 6.25 | 6.10 |
| | | | 3.92 | 7.25 | 8.00 | 0.00 | 0.00 | 13.50 |
| | | | 2.60 | 6.63 | 7.00 | 0.00 | 0.00 | 11.60 |
| | | | 2.72 | 7.25 | 6.50 | 0.00 | 0.00 | 9.20 |
| | | | 2.73 | 6.63 | 6.75 | 0.00 | 0.00 | 8.60 |
| | | | 2.60 | 6.50 | 7.00 | 0.00 | 0.00 | 8.50 |
| | | | 2.78 | 6.75 | 7.00 | 0.00 | 0.00 | 8.00 |
| | | | 2.94 | 7.13 | 6.50 | 0.00 | 0.00 | 10.20 |
| | | | 2.82 | 7.00 | 7.00 | 0.00 | 0.00 | 9.50 |
| | | | 3.22 | 7.00 | 7.50 | 0.00 | 0.00 | 11.10 |
| | | | 3.34 | 7.00 | 8.00 | 0.00 | 0.00 | 8.50 |
| | | | 3.20 | 7.00 | 7.50 | 0.00 | 3.75 | 8.90 |
| | | | 3.04 | 6.75 | 8.00 | 0.00 | 4.00 | 8.60 |
| | | | 3.30 | 7.25 | 7.25 | 0.00 | 0.00 | 10.70 |
| | | | 3.02 | 7.25 | 7.25 | 0.00 | 0.00 | 9.30 |
| | | | 2.82 | 6.50 | 7.50 | 4.25 | 7.75 | 9.70 |
| | | Avg. | 2.64 | 6.59 | 6.95 | 0.54 | 1.85 | 9.29 |
| | | St. Dev. | 0.54 | 0.50 | 0.51 | 1.40 | 2.60 | 1.71 |
| 24-Feb | TAM Dew Improved | 28 | 1.50 | 5.50 | 6.00 | 0.00 | 0.00 | 8.80 |
| | | | 1.50 | 5.25 | 6.00 | 0.00 | 4.00 | 7.40 |
| | | | 2.10 | 5.88 | 6.75 | 0.00 | 4.25 | 10.10 |
| | | | 1.00 | 4.75 | 5.50 | 0.00 | 3.25 | 5.40 |
| | | | 1.15 | 4.75 | 5.50 | 0.00 | 3.25 | 5.10 |
| | | | 1.33 | 5.25 | 5.50 | 0.00 | 0.00 | 6.70 |
| | | | 1.50 | 5.50 | 5.50 | 0.00 | 0.00 | 9.80 |
| | | | 0.98 | 5.00 | 5.25 | 0.00 | 4.25 | 6.20 |
| | | | 1.53 | 5.50 | 5.75 | 0.00 | 3.50 | 9.80 |
| | | | 1.24 | 5.00 | 5.50 | 0.00 | 4.25 | 7.10 |
| | | | 1.43 | 5.50 | 5.75 | 0.00 | 0.00 | 8.60 |
| | | | 1.57 | 5.63 | 5.75 | 0.00 | 0.00 | 9.70 |
| | | | 1.50 | 5.50 | 6.00 | 0.00 | 0.00 | 6.30 |
| | | | 1.88 | 6.00 | 6.50 | 4.00 | 6.50 | 7.20 |
| | | | 1.40 | 5.25 | 5.75 | 0.00 | 0.00 | 5.40 |
| | | | 1.40 | 5.25 | 6.00 | 0.00 | 3.75 | 7.00 |
| | | | 1.51 | 5.50 | 5.50 | 0.00 | 0.00 | 11.40 |
| | | | 1.82 | 6.00 | 6.00 | 0.00 | 0.00 | 7.40 |
| | | | 1.62 | 5.50 | 6.00 | 3.25 | 4.00 | 9.50 |
| | | | 1.45 | 5.50 | 5.63 | 0.00 | 0.00 | 7.00 |
| | | | 1.43 | 5.25 | 5.75 | 0.00 | 3.50 | 8.20 |
| | | | 1.68 | 5.75 | 6.25 | 0.00 | 3.00 | 7.40 |
| | | | 1.90 | 5.75 | 6.50 | 0.00 | 3.25 | 10.50 |
| | | | 1.70 | 5.50 | 6.25 | 0.00 | 3.50 | 9.60 |
| | | | 1.60 | 5.75 | 5.75 | 0.00 | 3.75 | 5.00 |
| | | | 2.20 | 6.38 | 6.25 | 0.00 | 0.00 | 11.50 |
| | | | 1.96 | 6.00 | 6.25 | 0.00 | 0.00 | 9.80 |
| | | | 1.80 | 5.63 | 6.50 | 3.75 | 6.00 | 6.60 |
| | | Avg. | 1.56 | 5.50 | 5.92 | 0.39 | 2.29 | 8.02 |
| | | St. Dev. | 0.29 | 0.38 | 0.38 | 1.16 | 2.14 | 1.91 |

### Example 6

### Demonstration of Harvest Maturity and Flesh Firmness at Fruit Separation from Stem

Test measurements were again carried out on melon fruit samples grown in the field in Woodland, Calif. in the summer of year 22 (Table 6). FIGS. 5-7 represent the averages from Table 6. For flesh firmness readings, melon fruit were cut in half and two penetrometer readings were taken with each size tip (8 mm and 11 mm). For the penetrometer readings, an 8 mm tip size penetrometer reading was taken on each cut melon half, then the tip was changed and an 11 mm tip size penetrometer reading was taken on each cut melon half. The penetrometer readings were taken on the melon flesh halfway between the interior cavity edge and the fruit skin. The average of the two readings for each tip size is represented in Table 6 and was used to determine averages for the melon variety types in FIGS. 5 and 6.

For Brix readings, each melon half was again tested, allowing the average of two readings to be used for each fruit. The average of the two readings is represented in Table 6 and was used to determine averages for the varieties represented in FIG. 7. A melon baller was used to sample a ball of melon flesh halfway between the interior cavity edge and the fruit skin, which was put into a garlic press and pressed for the juice sample. The juice sample was then applied to the refractometer for Brix measurement.

TABLE 6
Comparison of measurements for flesh firmness and Brix for parent, hybrid
and selected lines grown in Woodland, CA in the summer of year 22

| | | | LBF | LBF | LBF | LBF | LBF | LBF | | | |
|---|---|---|---|---|---|---|---|---|---|---|---|
| | | Fruit | 8 mm | 8 mm | 8 mm | 11 mm | 11 mm | 11 mm | Brix | Brix | Brix |
| Variety | Date | No. | read 1 | read 2 | Avg. | read 1 | read 2 | Avg. | read 1 | read 2 | Avg. |
| SVR03968074 | July 9 | 1 | 5 | 4 | 4.5 | 7 | 8.25 | 7.6 | 17.7 | 16 | 16.9 |
| | July 9 | 2 | 4.5 | 4 | 4.3 | 7.5 | 8 | 7.8 | 15.2 | 15.1 | 15.2 |
| | July 9 | 3 | 5.5 | 6.5 | 6.0 | 11 | 11.5 | 11.3 | 14.5 | 15.4 | 15.0 |
| | July 10 | 4 | 6 | 5.5 | 5.8 | 10.5 | 9.75 | 10.1 | 15.1 | 15.5 | 15.3 |
| | July 13 | 5 | 6 | 6.25 | 6.1 | 8.75 | 12.75 | 10.8 | 15.7 | 15.8 | 15.8 |
| | July 13 | 6 | 8.25 | 10.25 | 9.3 | 13.5 | 10.75 | 12.1 | 16 | 16 | 16.0 |
| | July 13 | 7 | 7.75 | 7 | 7.4 | 8.75 | 14.25 | 11.5 | 16 | 16.2 | 16.1 |
| | July 13 | 8 | 6.25 | 6.5 | 6.4 | 10.5 | 9.5 | 10.0 | 16.1 | 16.2 | 16.2 |
| | July 13 | 9 | 7.25 | 6.75 | 7.0 | 11 | 11.75 | 11.4 | 17.3 | 16.8 | 17.1 |
| | July 13 | 10 | 9.25 | 8 | 8.6 | 11 | 11 | 11.0 | 16.4 | 16.6 | 16.5 |
| | July 13 | 11 | 7.25 | 6.25 | 6.8 | 10.25 | 11.5 | 10.9 | 16.4 | 15.7 | 16.1 |
| | July 13 | 12 | 10.25 | 8.75 | 9.5 | 12.5 | 11.75 | 12.1 | 16.1 | 16.6 | 16.4 |
| | July 13 | 13 | 7 | 7 | 7.0 | 7.75 | 9.5 | 8.6 | 17 | 16.2 | 16.6 |
| | July 13 | 14 | 7.5 | 8.75 | 8.1 | 11 | 12 | 11.5 | 16 | 16.4 | 16.2 |
| | July 13 | 15 | 9.5 | 9 | 9.3 | 16.5 | 15.25 | 15.9 | 16.2 | 16.6 | 16.4 |
| | July 13 | 16 | 8.5 | 8 | 8.3 | 11.25 | 12.5 | 11.9 | 15.9 | 16.4 | 16.2 |
| | July 13 | 17 | 8.5 | 9.25 | 8.9 | 11.25 | 15 | 13.1 | 14.2 | 14.3 | 14.3 |
| | July 13 | 18 | 9.75 | 9.75 | 9.8 | 16.5 | 15.5 | 16.0 | 14.4 | 13.2 | 13.8 |
| | July 13 | 19 | 7.75 | 8.75 | 8.3 | 10.75 | 11 | 10.9 | 15.2 | 15 | 15.1 |
| | July 13 | 20 | 6.75 | 8 | 7.4 | 10.75 | 11.5 | 11.1 | 14.9 | 15.3 | 15.1 |
| | July 13 | 21 | 6.75 | 8.75 | 7.8 | 9 | 13 | 11.0 | 16.5 | 16.1 | 16.3 |
| | July 13 | 22 | 7.75 | 6.5 | 7.1 | 13.25 | 11.25 | 12.3 | 15.9 | 15 | 15.5 |
| | July 13 | 23 | 9.5 | 8.5 | 9.0 | 11.25 | 12 | 11.6 | 12.8 | 14 | 13.4 |
| | July 13 | 24 | 11 | 11.5 | 11.3 | 18 | 15.75 | 16.9 | 14.4 | 13.9 | 14.2 |
| | July 13 | 25 | 10.25 | 6.75 | 8.5 | 11.5 | 12.5 | 12.0 | 14.8 | 16 | 15.4 |
| | July 13 | 26 | 8.25 | 9.25 | 8.8 | 15 | 12.5 | 13.8 | 15.8 | 15.1 | 15.5 |
| | July 13 | 27 | 9.5 | 11.5 | 10.5 | 14 | 17.25 | 15.6 | 16.3 | 15.7 | 16.0 |
| | July 13 | 28 | 10.25 | 11 | 10.6 | 17.75 | 18 | 17.9 | 14.7 | 14.8 | 14.8 |
| | July 13 | 29 | 10 | 10.5 | 10.3 | 14.5 | 16.5 | 15.5 | 14.7 | 15.3 | 15.0 |
| | July 13 | 30 | 8.25 | 7.5 | 7.9 | 16.5 | 14 | 15.3 | 15.9 | 15.1 | 15.5 |
| | July 13 | 31 | 7.75 | 8 | 7.9 | 12.25 | 18.5 | 15.4 | 16.5 | 16.2 | 16.4 |
| | July 13 | 32 | 9.5 | 10.5 | 10.0 | 12 | 13 | 12.5 | 15.8 | 15.9 | 15.9 |
| | July 13 | 33 | 9 | 10 | 9.5 | 11 | 11.25 | 11.1 | 15.8 | 15.8 | 15.8 |
| | July 13 | 34 | 9.5 | 10.25 | 9.9 | 13.5 | 17.75 | 15.6 | 11.2 | 13 | 12.1 |
| | July 13 | 35 | 8 | 9.5 | 8.8 | 16 | 16.25 | 16.1 | 14 | 15.5 | 14.8 |
| | July 13 | 36 | 9 | 9 | 9.0 | 15.5 | 14.5 | 15.0 | 16.6 | 14.6 | 15.6 |
| | July 13 | 37 | 10 | 8.25 | 9.1 | 13.5 | 17 | 15.3 | 17 | 17.4 | 17.2 |
| | July 13 | 38 | 9.75 | 8.75 | 9.3 | 14 | 15.75 | 14.9 | 15.7 | 14.9 | 15.3 |
| | July 13 | 39 | 9.75 | 10 | 9.9 | 14.5 | 14 | 14.3 | 13.8 | 14.6 | 14.2 |
| | July 13 | 40 | 10.25 | 9.75 | 10.0 | 11.75 | 14 | 12.9 | 15.9 | 17.2 | 16.6 |
| | July 13 | 41 | 9 | 7.75 | 8.4 | 11.75 | 15.25 | 13.5 | 16.3 | 16.1 | 16.2 |
| | July 13 | 42 | 9.75 | 9.75 | 9.8 | 14.5 | 13.25 | 13.9 | 16.2 | 15.7 | 16.0 |
| | July 13 | 43 | 7.5 | 6.75 | 7.1 | 14.75 | 15.25 | 15.0 | 15.3 | 14.9 | 15.1 |
| | July 13 | 44 | 8.5 | 9.25 | 8.9 | 12.75 | 16.5 | 14.6 | 16.9 | 17.4 | 17.2 |
| | July 13 | 45 | 8 | 9.5 | 8.8 | 14.5 | 12.75 | 13.6 | 16 | 15.8 | 15.9 |
| | July 13 | 46 | 10.75 | 9.25 | 10.0 | 15.25 | 14.25 | 14.8 | 15.5 | 15.3 | 15.4 |
| | July 13 | 47 | 11.5 | 10.5 | 11.0 | 19.75 | 16.5 | 18.1 | 15.1 | 15.6 | 15.4 |
| | July 13 | 48 | 9.25 | 8 | 8.6 | 13.75 | 13.75 | 13.8 | 16.9 | 16.1 | 16.5 |
| | July 13 | 49 | 10.25 | 9.25 | 9.8 | 18.5 | 17.5 | 18.0 | 14.2 | 13.9 | 14.1 |
| | July 13 | 50 | 9 | 8.25 | 8.6 | 16.25 | 17 | 16.6 | 14.7 | 14.4 | 14.6 |
| | July 13 | 51 | 10.25 | 11.25 | 10.8 | 19 | 15.25 | 17.1 | 14.3 | 15.2 | 14.8 |
| | July 13 | 52 | 8.25 | 9.25 | 8.8 | 18 | 15.75 | 16.9 | 16.4 | 17.3 | 16.9 |
| Averages | | | | | 8.5 | | | 13.4 | | | 15.6 |
| SVR03968106 | July 10 | 1 | 7 | 6.5 | 6.8 | 9.5 | 9 | 9.3 | 9.7 | 8.5 | 9.1 |
| | July 10 | 2 | 6.5 | 5.25 | 5.9 | 10 | 10.75 | 10.4 | 8.8 | 8.7 | 8.8 |
| | July 10 | 3 | 5.5 | 4 | 4.8 | 8.5 | 8 | 8.3 | 11.8 | 13.1 | 12.5 |
| | July 10 | 4 | 6.25 | 6 | 6.1 | 9 | 9.25 | 9.1 | 8.7 | 8.1 | 8.4 |
| | July 13 | 5 | 5.5 | 7 | 6.3 | 14.25 | 11.25 | 12.8 | 16.6 | 14.8 | 15.7 |
| | July 13 | 6 | 6.5 | 8 | 7.3 | 9 | 9.5 | 9.3 | 14.7 | 14.6 | 14.7 |
| | July 13 | 7 | 8 | 7 | 7.5 | 8 | 9.25 | 8.6 | 15 | 15.2 | 15.1 |
| | July 13 | 8 | 6.25 | 6.25 | 6.3 | 10 | 10.75 | 10.4 | 15 | 15.1 | 15.1 |
| | July 13 | 9 | 6.5 | 7.5 | 7.0 | 10 | 10.5 | 10.3 | 14.1 | 13.5 | 13.8 |
| | July 13 | 10 | 7 | 6.5 | 6.8 | 10.5 | 10 | 10.3 | 13.1 | 12.9 | 13.0 |
| | July 13 | 11 | 8 | 7 | 7.5 | 10.5 | 10 | 10.3 | 13.8 | 13.4 | 13.6 |
| | July 13 | 12 | 5.25 | 6.75 | 6.0 | 7 | 7.5 | 7.3 | 13.3 | 12.1 | 12.7 |
| | July 13 | 13 | 5.5 | 5.5 | 5.5 | 10 | 10 | 10.0 | 14.5 | 14.2 | 14.4 |
| | July 13 | 14 | 5.75 | 5.75 | 5.8 | 8.5 | 8.25 | 8.4 | 13.2 | 12.6 | 12.9 |
| | July 13 | 15 | 6 | 7 | 6.5 | 10 | 9 | 9.5 | 12.7 | 12.4 | 12.6 |
| | July 13 | 16 | 5.5 | 5.5 | 5.5 | 9.25 | 8.5 | 8.9 | 13.5 | 13.9 | 13.7 |
| | July 13 | 17 | 7.5 | 7 | 7.3 | 11.5 | 9.75 | 10.6 | 13.3 | 13.8 | 13.6 |
| | July 14 | 18 | 10 | 9.25 | 9.6 | 9.5 | 11 | 10.3 | 15 | 12.3 | 13.7 |
| | July 14 | 19 | 9.25 | 9.75 | 9.5 | 9.5 | 9.75 | 9.6 | 11.9 | 11.6 | 11.8 |
| | July 14 | 20 | 9 | 8.75 | 8.9 | 14.25 | 15 | 14.6 | 12.7 | 14.8 | 13.8 |
| | July 14 | 21 | 10.25 | 8.5 | 9.4 | 16.25 | 16.25 | 16.3 | 13.8 | 12.8 | 13.3 |
| | July 14 | 22 | 8 | 7 | 7.5 | 9.5 | 12.25 | 10.9 | 15 | 15.5 | 15.3 |
| | July 14 | 23 | 7 | 8.5 | 7.8 | 10 | 9.75 | 9.9 | 12.7 | 10.2 | 11.5 |
| | July 14 | 24 | 7.25 | 8.5 | 7.9 | 13.75 | 14.75 | 14.3 | 9.3 | 10.9 | 10.1 |
| | July 14 | 25 | 7.25 | 7 | 7.1 | 12 | 13.25 | 12.6 | 13.2 | 13.2 | 13.2 |
| | July 14 | 26 | 11 | 9.5 | 10.3 | 12.5 | 21.25 | 16.9 | 12 | 12.7 | 12.4 |
| | July 14 | 27 | 7.25 | 8 | 7.6 | 9.75 | 14.5 | 12.1 | 13.8 | 14.5 | 14.2 |
| | July 14 | 28 | 8.5 | 7 | 7.8 | 13.5 | 12.25 | 12.9 | 12.6 | 14 | 13.3 |
| | July 14 | 29 | 7.5 | 8 | 7.8 | 11 | 12.25 | 11.6 | 11.9 | 12.4 | 12.2 |
| | July 14 | 30 | 15.25 | 11.5 | 13.4 | 15 | 18 | 16.5 | 12.5 | 11.7 | 12.1 |
| | July 14 | 31 | 11.5 | 9 | 10.3 | 14.5 | 13.25 | 13.9 | 13.6 | 13.5 | 13.6 |
| | July 14 | 32 | 11.75 | 10.25 | 11.0 | 17 | 15.5 | 16.3 | 12.5 | 13 | 12.8 |
| | July 14 | 33 | 9.5 | 10.5 | 10.0 | 14 | 15.5 | 14.8 | 14.1 | 15 | 14.6 |
| | July 14 | 34 | 10.5 | 11 | 10.8 | 14.75 | 13.5 | 14.1 | 15.3 | 12.3 | 13.8 |
| | July 14 | 35 | 9 | 8 | 8.5 | 12 | 13.25 | 12.6 | 12.3 | 11 | 11.7 |
| | July 14 | 36 | 9 | 10.75 | 9.9 | 13.5 | 14.25 | 13.9 | 10.7 | 10.7 | 10.7 |
| | July 14 | 37 | 8 | 9.25 | 8.6 | 14.5 | 12.75 | 13.6 | 12.2 | 14.8 | 13.5 |
| | July 14 | 38 | 6.5 | 8.5 | 7.5 | 12.5 | 10 | 11.3 | 13.7 | 13.5 | 13.6 |
| | July 14 | 39 | 8.5 | 8 | 8.3 | 12.5 | 12 | 12.3 | 13.7 | 14.2 | 14.0 |
| | July 14 | 40 | 8.5 | 7 | 7.8 | 11.75 | 10 | 10.9 | 15.2 | 15 | 15.1 |
| | July 14 | 41 | 7.75 | 16 | 11.9 | 14.5 | 14 | 14.3 | 14.5 | 14 | 14.3 |
| | July 14 | 42 | 8.5 | 8.25 | 8.4 | 12.75 | 14 | 13.4 | 14.9 | 16.1 | 15.5 |
| | July 14 | 43 | 11.5 | 13 | 12.3 | 12.5 | 13.5 | 13.0 | 14.4 | 14.5 | 14.5 |
| | July 14 | 44 | 7.5 | 7.5 | 7.5 | 13 | 9.5 | 11.3 | 13.4 | 14.2 | 13.8 |
| | July 14 | 45 | 7 | 8.75 | 7.9 | 9.75 | 11 | 10.4 | 13.5 | 15.3 | 14.4 |
| | July 14 | 46 | 7.5 | 7 | 7.3 | 10.75 | 11.5 | 11.1 | 15 | 13.1 | 14.1 |
| | July 14 | 47 | 7.75 | 9 | 8.4 | 15 | 11.5 | 13.3 | 14.1 | 14.4 | 14.3 |
| | July 14 | 48 | 8 | 6.5 | 7.3 | 14 | 8.5 | 11.3 | 14.2 | 12.9 | 13.6 |
| | July 14 | 49 | 6.5 | 7 | 6.8 | 10.5 | 9.75 | 10.1 | 13.1 | 13.2 | 13.2 |
| | July 14 | 50 | 6.25 | 5 | 5.6 | 8.75 | 7.5 | 8.1 | 13.2 | 13.6 | 13.4 |
| | July 14 | 51 | 8.75 | 9 | 8.9 | 11.75 | 10.75 | 11.3 | 12.3 | 12.6 | 12.5 |
| | July 14 | 52 | 7 | 7.75 | 7.4 | 14 | 12 | 13.0 | 15.5 | 14.4 | 15.0 |
| | July 14 | 53 | 7.25 | 9.25 | 8.3 | 12.5 | 14.75 | 13.6 | 14.1 | 14 | 14.1 |
| | July 14 | 54 | 8 | 8.25 | 8.1 | 14.25 | 10.25 | 12.3 | 14.2 | 14.9 | 14.6 |
| | July 14 | 55 | 6.75 | 7.75 | 7.3 | 11.75 | 10.75 | 11.3 | 14.1 | 12.6 | 13.4 |
| | July 14 | 56 | 6.75 | 7.75 | 7.3 | 10.25 | 10 | 10.1 | 14 | 13.9 | 14.0 |
| | July 14 | 57 | 8.75 | 6.75 | 7.8 | 10.5 | 10.5 | 10.5 | 14.3 | 15.2 | 14.8 |
| | July 14 | 58 | 7 | 7.25 | 7.1 | 11 | 10 | 10.5 | 15.6 | 15.1 | 15.4 |
| | July 14 | 59 | 8 | 8.25 | 8.1 | 11.5 | 11.25 | 11.4 | 15.1 | 13.8 | 14.5 |
| | July 14 | 60 | 9 | 6.5 | 7.8 | 12 | 10 | 11.0 | 13.3 | 13.9 | 13.6 |
| | July 14 | 61 | 6.25 | 7 | 6.6 | 10 | 9.75 | 9.9 | 14.2 | 13.9 | 14.1 |
| | July 14 | 62 | 7 | 7.25 | 7.1 | 12.5 | 9.75 | 11.1 | 12.7 | 12.1 | 12.4 |
| | July 14 | 63 | 6.5 | 7 | 6.8 | 11.75 | 12.5 | 12.1 | 14.4 | 15.3 | 14.9 |
| | July 14 | 64 | 9.5 | 7.25 | 8.4 | 16 | 13 | 14.5 | 14.7 | 14.3 | 14.5 |
| | July 14 | 65 | 9 | 7 | 8.0 | 13 | 13.5 | 13.3 | 14.8 | 15.1 | 15.0 |
| | July 14 | 66 | 10.25 | 10 | 10.1 | 16 | 16 | 16.0 | 14.1 | 13.8 | 14.0 |
| | July 14 | 67 | 9.75 | 7.5 | 8.6 | 15.5 | 14 | 14.8 | 14.3 | 15 | 14.7 |
| | July 14 | 68 | 8.75 | 8.75 | 8.8 | 13.5 | 12.75 | 13.1 | 15.5 | 14.3 | 14.9 |
| Averages | | | | | 8.0 | | | 11.8 | | | 13.5 |
| HDG39-2023AN | July 13 | 5 | 6.5 | 7 | 6.8 | 10.5 | 8.75 | 9.6 | 15.2 | 15.5 | 15.4 |
| | July 13 | 6 | 7 | 10.25 | 8.6 | 13.25 | 13.5 | 13.4 | 15.7 | 14.8 | 15.3 |
| | July 13 | 7 | 6.5 | 6.25 | 6.4 | 12.75 | 10.25 | 11.5 | 16.5 | 15.9 | 16.2 |
| | July 13 | 8 | 5.5 | 5.75 | 5.6 | 11.75 | 12.25 | 12.0 | 15.3 | 14 | 14.7 |
| | July 13 | 9 | 7.5 | 8.5 | 8.0 | 12.75 | 9.25 | 11.0 | 14 | 15.8 | 14.9 |
| | July 13 | 10 | 9.5 | 7.75 | 8.6 | 16 | 12.75 | 14.4 | 16.1 | 16.4 | 16.3 |
| | July 13 | 11 | 6.25 | 7 | 6.6 | 13.25 | 13 | 13.1 | 15.7 | 14.5 | 15.1 |
| | July 13 | 12 | 9.75 | 10.25 | 10.0 | 17.5 | 18.5 | 18.0 | 16.1 | 14.9 | 15.5 |
| | July 13 | 13 | 10 | 11.25 | 10.6 | 15 | 14.25 | 14.6 | 15.6 | 15 | 15.3 |
| | July 13 | 14 | 6 | 6.75 | 6.4 | 13 | 14.75 | 13.9 | 14.5 | 14.5 | 14.5 |
| | July 13 | 15 | 8.5 | 10.25 | 9.4 | 12.5 | 15.75 | 14.1 | 15 | 8.6 | 11.8 |
| | July 13 | 16 | 8 | 7 | 7.5 | 11.5 | 12.25 | 11.9 | 15.3 | 14.5 | 14.9 |
| | July 13 | 17 | 7.5 | 8.5 | 8.0 | 17.75 | 17.75 | 17.8 | 13.2 | 14.4 | 13.8 |
| | July 13 | 18 | 9 | 8 | 8.5 | 14.25 | 11.25 | 12.8 | 13.4 | 14.3 | 13.9 |
| | July 13 | 19 | 8 | 9 | 8.5 | 14.25 | 12.5 | 13.4 | 16.3 | 15.8 | 16.1 |
| | July 13 | 20 | 6 | 8.5 | 7.3 | 11.75 | 15.5 | 13.6 | 14.9 | 15.2 | 15.1 |
| | July 13 | 21 | 7.5 | 7.5 | 7.5 | 15.25 | 15.25 | 15.3 | 14 | 13.3 | 13.7 |
| | July 13 | 22 | 9.5 | 8.25 | 8.9 | 16.75 | 12.75 | 14.8 | 12.7 | 14.9 | 13.8 |
| | July 13 | 23 | 8 | 7.75 | 7.9 | 16.75 | 11.25 | 14.0 | 14.4 | 14.6 | 14.5 |
| | July 13 | 24 | 6.75 | 7.75 | 7.3 | 11.75 | 14.25 | 13.0 | 12.9 | 12.9 | 12.9 |
| | July 13 | 25 | 8.75 | 8.5 | 8.6 | 11.75 | 13.25 | 12.5 | 14.3 | 14.2 | 14.3 |
| | July 13 | 26 | 4.75 | 6.5 | 5.6 | 9 | 9 | 9.0 | 17 | 16.3 | 16.7 |
| | July 13 | 27 | 7.5 | 9.25 | 8.4 | 15.25 | 11.25 | 13.3 | 16.3 | 16.4 | 16.4 |
| | July 13 | 28 | 8.75 | 6.75 | 7.8 | 13.5 | 12.25 | 12.9 | 16.8 | 16.9 | 16.9 |
| | July 13 | 29 | 9.25 | 9.75 | 9.5 | 13.5 | 13.5 | 13.5 | 16.8 | 16.3 | 16.6 |
| | July 13 | 30 | 8.25 | 8 | 8.1 | 13.25 | 12.25 | 12.8 | 15 | 15.4 | 15.2 |
| | July 13 | 31 | 6.75 | 8.25 | 7.5 | 14.25 | 13.5 | 13.9 | 15.1 | 14.6 | 14.9 |
| | July 13 | 32 | 12.75 | 9 | 10.9 | 15.75 | 15 | 15.4 | 16.6 | 15.5 | 16.1 |
| | July 13 | 33 | 8.25 | 9 | 8.6 | 10.75 | 15 | 12.9 | 14.4 | 15.2 | 14.8 |
| | July 13 | 34 | 10.75 | 9.5 | 10.1 | 13.25 | 15 | 14.1 | 15.3 | 14.6 | 15.0 |
| | July 13 | 35 | 8.5 | 9.75 | 9.1 | 13.5 | 14.25 | 13.9 | 15.8 | 14.7 | 15.3 |
| | July 13 | 36 | 7.5 | 6 | 6.8 | 17.75 | 14.5 | 16.1 | 16.8 | 16.7 | 16.8 |
| | July 13 | 37 | 10.25 | 10.25 | 10.3 | 17 | 18 | 17.5 | 15.6 | 14.1 | 14.9 |
| | July 13 | 38 | 8.5 | 7.75 | 8.1 | 12 | 15 | 13.5 | 14.8 | 15.6 | 15.2 |
| | July 13 | 39 | 5.5 | 6.25 | 5.9 | 11.5 | 9.25 | 10.4 | 15.5 | 14.6 | 15.1 |
| | July 13 | 40 | 9.25 | 7.25 | 8.3 | 12.75 | 10.75 | 11.8 | 14.1 | 13.9 | 14.0 |
| | July 13 | 41 | 7.75 | 9 | 8.4 | 13.75 | 15 | 14.4 | 13.7 | 16.8 | 15.3 |
| | July 13 | 42 | 10.25 | 9.5 | 9.9 | 15.75 | 14.5 | 15.1 | 15 | 16.5 | 15.8 |
| | July 13 | 43 | 7.5 | 7.5 | 7.5 | 12.75 | 13.75 | 13.3 | 15.2 | 14.4 | 14.8 |
| | July 13 | 44 | 8.25 | 8 | 8.1 | 12.5 | 11.5 | 12.0 | 16.2 | 16.1 | 16.2 |
| | July 13 | 45 | 8 | 7.5 | 7.8 | 11.25 | 10 | 10.6 | 15.3 | 14.7 | 15.0 |
| | July 13 | 46 | 10.5 | 10.75 | 10.6 | 13 | 14.75 | 13.9 | 14.2 | 15.1 | 14.7 |
| | July 13 | 47 | 9.75 | 8.25 | 9.0 | 13.25 | 11 | 12.1 | 13.6 | 15.2 | 14.4 |
| | July 13 | 48 | 5.25 | 5.25 | 5.3 | 10.5 | 8.75 | 9.6 | 13.8 | 16.2 | 15.0 |
| | July 13 | 49 | 5.25 | 7.25 | 6.3 | 8 | 8.25 | 8.1 | 16 | 14.1 | 15.1 |
| | July 13 | 50 | 10 | 11.25 | 10.6 | 10.5 | 14 | 12.3 | 14.3 | 15.6 | 15.0 |
| | July 13 | 51 | 8.25 | 8 | 8.1 | 11.25 | 11.5 | 11.4 | 15.6 | 15.9 | 15.8 |
| | July 13 | 52 | 8 | 5.25 | 6.6 | 10.5 | 8.75 | 9.6 | 9.9 | 10.9 | 10.4 |
| | July 13 | 53 | 8 | 7.75 | 7.9 | 12.25 | 11.75 | 12.0 | 14.7 | 15.7 | 15.2 |
| | July 13 | 54 | 7.75 | 7.25 | 7.5 | 12.75 | 10.5 | 11.6 | 13.2 | 8.5 | 10.9 |
| Averages | | | | | 8.1 | | | 13.0 | | | 14.9 |
| HDG39-2021AN | July 13 | 1 | 6 | 5 | 5.5 | 11.5 | 10 | 10.8 | 13.8 | 13.8 | 13.8 |
| | July 13 | 2 | 5.25 | 7 | 6.1 | 11 | 14 | 12.5 | 13 | 14.1 | 13.6 |
| | July 13 | 3 | 6.5 | 9.5 | 8.0 | 12 | 14 | 13.0 | 13.2 | 13.3 | 13.3 |
| | July 13 | 4 | 6.75 | 10 | 8.4 | 10.75 | 9.25 | 10.0 | 12.3 | 14.1 | 13.2 |
| | July 14 | 5 | 9 | 9 | 9.0 | 11.25 | 11.25 | 11.3 | 14.5 | 13.8 | 14.2 |
| | July 14 | 6 | 10 | 9.75 | 9.9 | 14.5 | 13 | 13.8 | 13.1 | 12.4 | 12.8 |
| | July 14 | 7 | 9.5 | 9.25 | 9.4 | 15 | 16 | 15.5 | 12.6 | 11.2 | 11.9 |
| | July 14 | 8 | 11.25 | 12.5 | 11.9 | 16 | 13.5 | 14.8 | 12.4 | 11.2 | 11.8 |
| | July 14 | 9 | 8 | 9.5 | 8.8 | 12.75 | 14 | 13.4 | 13.1 | 13.7 | 13.4 |
| | July 14 | 10 | 8.75 | 7.5 | 8.1 | 11 | 12 | 11.5 | 13.4 | 14.4 | 13.9 |
| | July 14 | 11 | 6.75 | 8.25 | 7.5 | 13.25 | 11.75 | 12.5 | 13.3 | 13.4 | 13.4 |
| | July 14 | 12 | 6 | 7.25 | 6.6 | 14 | 15.5 | 14.8 | 14.4 | 14.8 | 14.6 |
| | July 14 | 13 | 8.5 | 8 | 8.3 | 13.75 | 12.5 | 13.1 | 14.6 | 13.3 | 14.0 |
| | July 14 | 14 | 11.5 | 11 | 11.3 | 15 | 13.5 | 14.3 | 11.1 | 11.4 | 11.3 |
| | July 14 | 15 | 7.25 | 7 | 7.1 | 9 | 11 | 10.0 | 13.8 | 13.9 | 13.9 |
| | July 14 | 16 | 7.75 | 9.75 | 8.8 | 14 | 12.5 | 13.3 | 13.5 | 12.2 | 12.9 |
| | July 14 | 17 | 10.5 | 9 | 9.8 | 13.25 | 14 | 13.6 | 14.1 | 11.7 | 12.9 |
| | July 14 | 18 | 8 | 9 | 8.5 | 15.25 | 12.25 | 13.8 | 12.8 | 12.6 | 12.7 |
| | July 14 | 19 | 7.75 | 8.75 | 8.3 | 9.75 | 13 | 11.4 | 12.4 | 15 | 13.7 |
| | July 14 | 20 | 9.25 | 8.5 | 8.9 | 14.5 | 14.25 | 14.4 | 14.2 | 13.1 | 13.7 |
| | July 14 | 21 | 11 | 9.5 | 10.3 | 12.5 | 12 | 12.3 | 13.3 | 12.5 | 12.9 |
| | July 14 | 22 | 9.75 | 9.5 | 9.6 | 13.25 | 12 | 12.6 | 13.9 | 14.1 | 14.0 |
| | July 14 | 23 | 9 | 8.75 | 8.9 | 11.5 | 14 | 12.8 | 13.9 | 11.4 | 12.7 |
| | July 14 | 24 | 10.75 | 11.75 | 11.3 | 18.5 | 14.75 | 16.6 | 10.5 | 9.8 | 10.2 |
| | July 14 | 25 | 10.5 | 9.75 | 10.1 | 15 | 16 | 15.5 | 13.6 | 14.1 | 13.9 |
| | July 14 | 26 | 11 | 10 | 10.5 | 16 | 15 | 15.5 | 12.6 | 11.9 | 12.3 |
| | July 14 | 27 | 8.75 | 9.5 | 9.1 | 12.75 | 15 | 13.9 | 13.2 | 14.2 | 13.7 |
| | July 14 | 28 | 10 | 9 | 9.5 | 13.5 | 14.25 | 13.9 | 13.9 | 13.9 | 13.9 |
| | July 14 | 29 | 11 | 9 | 10.0 | 15.5 | 12.5 | 14.0 | 15.3 | 14.5 | 14.9 |
| | July 14 | 30 | 10 | 9 | 9.5 | 13.75 | 12.25 | 13.0 | 14 | 14 | 14.0 |
| | July 14 | 31 | 9.75 | 8.75 | 9.3 | 12.5 | 12 | 12.3 | 13.7 | 13.9 | 13.8 |
| | July 14 | 32 | 9 | 8.75 | 8.9 | 12.75 | 12.75 | 12.8 | 13.2 | 13.3 | 13.3 |
| | July 14 | 33 | 12.75 | 9 | 10.9 | 16.5 | 15.25 | 15.9 | 12.3 | 11.7 | 12.0 |
| | July 14 | 34 | 9.75 | 9 | 9.4 | 13.25 | 12 | 12.6 | 13.2 | 12.9 | 13.1 |
| | July 14 | 35 | 8.75 | 8.25 | 8.5 | 11.5 | 13 | 12.3 | 13.6 | 12.8 | 13.2 |
| | July 14 | 36 | 9.5 | 8 | 8.8 | 12 | 12.25 | 12.1 | 13 | 11.8 | 12.4 |
| | July 14 | 37 | 17.5 | 10.75 | 14.1 | 16 | 15.75 | 15.9 | 14.8 | 13.8 | 14.3 |
| | July 14 | 38 | 10 | 9 | 9.5 | 16.75 | 16 | 16.4 | 12.8 | 13.8 | 13.3 |
| | July 14 | 39 | 9.5 | 10 | 9.8 | 12.5 | 12 | 12.3 | 14.6 | 13.6 | 14.1 |
| | July 14 | 40 | 9 | 8.75 | 8.9 | 13.5 | 13 | 13.3 | 12.9 | 12 | 12.5 |
| | July 14 | 41 | 9.25 | 7.75 | 8.5 | 14 | 13 | 13.5 | 13.7 | 14.3 | 14.0 |
| | July 14 | 42 | 7.75 | 9 | 8.4 | 14 | 11 | 12.5 | 12.9 | 13.2 | 13.1 |
| | July 14 | 43 | 9 | 9 | 9.0 | 11.5 | 12 | 11.8 | 13.3 | 13.6 | 13.5 |
| | July 14 | 44 | 9.5 | 8.25 | 8.9 | 14.5 | 11.5 | 13.0 | 13.5 | 12.7 | 13.1 |
| | July 14 | 45 | 10.25 | 10.75 | 10.5 | 15 | 12.25 | 13.6 | 13 | 12.8 | 12.9 |
| | July 14 | 46 | 8.5 | 9.5 | 9.0 | 12.5 | 11 | 11.8 | 12.2 | 12.3 | 12.3 |
| | July 14 | 47 | 8.75 | 8.75 | 8.8 | 12 | 13.25 | 12.6 | 11.8 | 12 | 11.9 |
| | July 14 | 48 | 9.25 | 7.5 | 8.4 | 13.75 | 12 | 12.9 | 13.5 | 14.1 | 13.8 |
| | July 14 | 49 | 10.25 | 8.5 | 9.4 | 13.75 | 16.5 | 15.1 | 15.1 | 14.2 | 14.7 |
| | July 14 | 50 | 6.25 | 10.5 | 8.4 | 12 | 14 | 13.0 | 12.4 | 13.2 | 12.8 |
| | July 14 | 51 | 7.5 | 8.25 | 7.9 | 11.25 | 13.25 | 12.3 | 11.8 | 11 | 11.4 |
| | July 14 | 52 | 5.5 | 6.75 | 6.1 | 14 | 12.5 | 13.3 | 13 | 11.7 | 12.4 |
| | July 14 | 53 | 7 | 7 | 7.0 | 10.25 | 11.25 | 10.8 | 13.7 | 12.8 | 13.3 |
| | July 14 | 54 | 8 | 8.75 | 8.4 | 12.5 | 14 | 13.3 | 12.3 | 12.5 | 12.4 |
| | July 14 | 55 | 8.75 | 9.75 | 9.3 | 13.5 | 15 | 14.3 | 11.9 | 13.1 | 12.5 |
| | July 14 | 56 | 9.75 | 9.75 | 9.8 | 13 | 15.25 | 14.1 | 14 | 12 | 13.0 |
| | July 14 | 57 | 9 | 7.5 | 8.3 | 14 | 13.5 | 13.8 | 13.7 | 13.6 | 13.7 |
| | July 14 | 58 | 8.75 | 7 | 7.9 | 12 | 14 | 13.0 | 14 | 13.7 | 13.9 |
| | July 14 | 59 | 8.25 | 9 | 8.6 | 15.25 | 15 | 15.1 | 12.9 | 12.7 | 12.8 |
| | July 14 | 60 | 8 | 8 | 8.0 | 12.5 | 13 | 12.8 | 12.4 | 12.7 | 12.6 |
| | July 14 | 61 | 9 | 11 | 10.0 | 11 | 13.75 | 12.4 | 14.8 | 12.7 | 13.8 |
| | July 14 | 62 | 7.25 | 8 | 7.6 | 18.5 | 15 | 16.8 | 13.6 | 11.6 | 12.6 |
| Averages | | | | | 8.9 | | | 13.3 | | | 13.1 |
| TAM Dew Improved | July 28 | 1 | 0 | 3.5 | 1.8 | 5 | 4.5 | 4.8 | 13.6 | 13.9 | 13.8 |
| | July 28 | 2 | 0 | 3 | 1.5 | 4.75 | 0 | 2.4 | 14.3 | 14.5 | 14.4 |
| | July 28 | 3 | 0 | 0 | 0.0 | 3.5 | 0 | 1.8 | 13.3 | 13.8 | 13.6 |
| | July 28 | 4 | 3 | 3 | 3.0 | 4 | 3.75 | 3.9 | 7.8 | 13.2 | 10.5 |
| | July 28 | 5 | 0 | 3.5 | 1.8 | 1 | 4.5 | 2.8 | 14.1 | 14.2 | 14.2 |
| | July 28 | 6 | 4.5 | 4.25 | 4.4 | 9.5 | 7.25 | 8.4 | 15.6 | 15.6 | 15.6 |
| | July 28 | 7 | 3.75 | 3.75 | 3.8 | 7.5 | 7 | 7.3 | 14.5 | 15.7 | 15.1 |
| | July 28 | 8 | 3 | 4 | 3.5 | 4 | 6 | 5.0 | 15.8 | 3.7 | 9.8 |
| | July 28 | 9 | 3.75 | 3.5 | 3.6 | 5.25 | 7 | 6.1 | 16.9 | 15 | 16.0 |
| | July 28 | 10 | 0 | 0 | 0.0 | 3.25 | 3 | 3.1 | 14.5 | 13.8 | 14.2 |
| | July 28 | 11 | 3.25 | 0 | 1.6 | 4.75 | 4 | 4.4 | 13.1 | 14.8 | 14.0 |
| | July 28 | 12 | 0 | 0 | 0.0 | 3 | 0 | 1.5 | 15.8 | 15.4 | 15.6 |
| | July 28 | 13 | 3.25 | 3 | 3.1 | 4 | 3.5 | 3.8 | 16 | 16.4 | 16.2 |
| | July 28 | 14 | 3 | 3 | 3.0 | 0 | 0 | 0.0 | 15.3 | 14.6 | 15.0 |
| | July 28 | 15 | 4.25 | 4 | 4.1 | 5.75 | 4.25 | 5.0 | 14.1 | 15.9 | 15.0 |
| | July 28 | 16 | 3 | 3.25 | 3.1 | 5 | 3.5 | 4.3 | 16.9 | 16.5 | 16.7 |
| | July 28 | 17 | 0 | 0 | 0.0 | 4 | 3 | 3.5 | 14.1 | 15.8 | 15.0 |
| | July 28 | 18 | 3.75 | 3 | 3.4 | 5 | 4.5 | 4.8 | 14.5 | 15.8 | 15.2 |
| | July 28 | 19 | 0 | 3 | 1.5 | 4.5 | 4.25 | 4.4 | 15.5 | 15.5 | 15.5 |
| | July 28 | 20 | 5.75 | 4.5 | 5.1 | 5.5 | 6.25 | 5.9 | 15.4 | 14.6 | 15.0 |
| | July 28 | 21 | 4.5 | 3.5 | 4.0 | 6 | 6 | 6.0 | 16.3 | 16.6 | 16.5 |
| | July 28 | 22 | 3 | 3 | 3.0 | 3.75 | 5.25 | 4.5 | 15.5 | 14.8 | 15.2 |
| | July 28 | 23 | 3 | 3.75 | 3.4 | 5 | 5.5 | 5.3 | 15 | 14.7 | 14.9 |
| | July 28 | 24 | 3.25 | 3 | 3.1 | 4.5 | 5 | 4.8 | 15.6 | 16 | 15.8 |
| | July 28 | 25 | 0 | 0 | 0.0 | 0 | 0 | 0.0 | 14.9 | 14.7 | 14.8 |
| | July 28 | 26 | 0 | 4 | 2.0 | 6.5 | 4 | 5.3 | 15 | 14.9 | 15.0 |
| | July 28 | 27 | 3.5 | 0 | 1.8 | 4.25 | 3 | 3.6 | 15.8 | 11.9 | 13.9 |
| | July 28 | 28 | 0 | 0 | 0.0 | 4 | 4.25 | 4.1 | 11 | 11.5 | 11.3 |
| | July 28 | 29 | 0 | 3 | 1.5 | 4.5 | 3 | 3.8 | 11.7 | 11.9 | 11.8 |
| | July 28 | 30 | 0 | 3 | 1.5 | 3 | 4 | 3.5 | 12.3 | 12.7 | 12.5 |
| | July 28 | 31 | 3 | 4.25 | 3.6 | 4.5 | 4.5 | 4.5 | 13.2 | 12.6 | 12.9 |
| | July 28 | 32 | 3.25 | 0 | 1.6 | 3.75 | 3.5 | 3.6 | 16.3 | 13.4 | 14.9 |
| | July 28 | 33 | 3.5 | 4 | 3.8 | 4.75 | 5.75 | 5.3 | 14.5 | 13.6 | 14.1 |
| | July 28 | 34 | 3.75 | 3 | 3.4 | 3.5 | 4.25 | 3.9 | 13.6 | 13.3 | 13.5 |
| | July 28 | 35 | 4.75 | 3.5 | 4.1 | 7.5 | 5.25 | 6.4 | 14 | 16.3 | 15.2 |
| | July 28 | 36 | 0 | 0 | 0.0 | 4.5 | 4 | 4.3 | 16 | 15.9 | 16.0 |
| | July 28 | 37 | 3 | 3.25 | 3.1 | 4.25 | 4.75 | 4.5 | 16 | 16 | 16.0 |
| | July 28 | 38 | 4 | 3 | 3.5 | 5.25 | 4.5 | 4.9 | 14.4 | 14.8 | 14.6 |
| Averages | | | | | 2.4 | | | 4.2 | | | 14.4 |
| Haley | July 13 | 1 | 12.5 | 9 | 10.8 | 13.75 | 13.75 | 13.8 | 13.5 | 12.8 | 13.2 |
| | July 28 | 2 | 0 | 0 | 0.0 | 3 | 3.25 | 3.1 | 16.4 | 16.6 | 16.5 |
| | July 28 | 3 | 3.25 | 3.75 | 3.5 | 4.5 | 5.5 | 5.0 | 16.1 | 15.6 | 15.9 |
| | July 28 | 4 | 3.25 | 3 | 3.1 | 3.5 | 4.5 | 4.0 | 16 | 16.1 | 16.1 |
| | July 28 | 5 | 3.75 | 3 | 3.4 | 5.75 | 6 | 5.9 | | 16.4 | 16.4 |
| | July 28 | 6 | 0 | 0 | 0.0 | 0 | 0 | 0.0 | 15.7 | 15.8 | 15.8 |
| | July 28 | 7 | 0 | 0 | 0.0 | 3.75 | 4.25 | 4.0 | 15.3 | 14.7 | 15.0 |
| | July 28 | 8 | 3 | 3.5 | 3.3 | 5.5 | 5 | 5.3 | 16.1 | 16.5 | 16.3 |
| | July 28 | 9 | 4 | 3.5 | 3.8 | 5 | 4 | 4.5 | 14.7 | 14.6 | 14.7 |
| | July 28 | 10 | 0 | 0 | 0.0 | 3.5 | 4.5 | 4.0 | 16.7 | 16.6 | 16.7 |
| | July 2 8 | 11 | 0 | 3 | 1.5 | 3 | 3.25 | 3.1 | 16.5 | 16.7 | 16.6 |
| | July 28 | 12 | 4 | 4 | 4.0 | 6.25 | 7 | 6.6 | 16.2 | 15.6 | 15.9 |
| | July 28 | 13 | 3.25 | 4 | 3.6 | 4.75 | 5 | 4.9 | 16.6 | 16.2 | 16.4 |
| | July 28 | 14 | 4 | 3 | 3.5 | 6 | 4.5 | 5.3 | 15.8 | 15.1 | 15.5 |
| | July 28 | 15 | 0 | 3 | 1.5 | 4 | 4.25 | 4.1 | 16.2 | 15.5 | 15.9 |
| | July 28 | 16 | 3 | 3.75 | 3.4 | 4.75 | 4.75 | 4.8 | 16.3 | 15.9 | 16.1 |
| | July 28 | 17 | 3.25 | 3 | 3.1 | 5 | 4.5 | 4.8 | 11.5 | 16.4 | 14.0 |
| | July 28 | 18 | 4.25 | 3 | 3.6 | 7 | 7 | 7.0 | 16.3 | 15.9 | 16.1 |
| | July 2 8 | 19 | 4 | 4.25 | 4.1 | 5.5 | 5.25 | 5.4 | 15.5 | 14.6 | 15.1 |
| | July 28 | 20 | 0 | 3 | 1.5 | 4.25 | 0 | 2.1 | 15.4 | 15.9 | 15.7 |
| | July 28 | 21 | 3.75 | 3 | 3.4 | 5.5 | 6 | 5.8 | 15.6 | 15.8 | 15.7 |
| | July 28 | 22 | 4 | 3.25 | 3.6 | 6.5 | 6 | 6.3 | 16.9 | 16.7 | 16.8 |
| | July 28 | 23 | 4.25 | 3.5 | 3.9 | 5.5 | 5 | 5.3 | 15.2 | 15.4 | 15.3 |
| | July 28 | 24 | 3 | 3.5 | 3.3 | 3.5 | 4.75 | 4.1 | 14.6 | 14.6 | 14.6 |
| | July 28 | 25 | 3.25 | 3.5 | 3.4 | 5 | 4 | 4.5 | 16.4 | 16.3 | 16.4 |
| | July 28 | 26 | 3.5 | 3.5 | 3.5 | 4.25 | 4 | 4.1 | 15.9 | 16.5 | 16.2 |
| | July 28 | 27 | 3.25 | 3.75 | 3.5 | 5.25 | 4.5 | 4.9 | 15.8 | 14 | 14.9 |
| | July 28 | 28 | 3 | 4.25 | 3.6 | 5.75 | 5.25 | 5.5 | 14.4 | 14.5 | 14.5 |
| | July 28 | 29 | 3.25 | 3 | 3.1 | 5.25 | 4 | 4.6 | 13 | 13.5 | 13.3 |
| | July 28 | 30 | 4 | 3.25 | 3.6 | 4.75 | 3.5 | 4.1 | 14.1 | 14.4 | 14.3 |
| | July 28 | 31 | 3 | 4.25 | 3.6 | 5 | 4 | 4.5 | 15 | 14.7 | 14.9 |
| | July 28 | 32 | 4.25 | 4 | 4.1 | 7.75 | 6 | 6.9 | 14.4 | 15.5 | 15.0 |
| | July 28 | 33 | 3.25 | 3.5 | 3.4 | 4 | 4 | 4.0 | 16.1 | 15.5 | 15.8 |
| | July 28 | 34 | 4 | 4.25 | 4.1 | 5.5 | 5.5 | 5.5 | 16.3 | 17.1 | 16.7 |
| | July 28 | 35 | 3 | 3 | 3.0 | 5.75 | 4.75 | 5.3 | 16.5 | 15.8 | 16.2 |
| | July 28 | 36 | 3 | 4 | 3.5 | 4 | 4 | 4.0 | 16 | 14.8 | 15.4 |
| | July 28 | 37 | 0 | 0 | 0.0 | 0 | 3 | 1.5 | 14.6 | 15 | 14.8 |
| | July 28 | 38 | 3.75 | 3.25 | 3.5 | 5.25 | 5 | 5.1 | 15.5 | 16.1 | 15.8 |
| | July 28 | 39 | 3.5 | 3.25 | 3.4 | 5.75 | 5.25 | 5.5 | 14.9 | 16.4 | 15.7 |
| | July 28 | 40 | 3 | 3 | 3.0 | 5.25 | 4.75 | 5.0 | 14.1 | 16.1 | 15.1 |
| | July 28 | 41 | 3.25 | 3.5 | 3.4 | 4.5 | 4.75 | 4.6 | 16.4 | 16.9 | 16.7 |
| | July 28 | 42 | 4.25 | 3.5 | 3.9 | 6 | 4.5 | 5.3 | 15.9 | 16.4 | 16.2 |
| | July 28 | 43 | 3.75 | 5.25 | 4.5 | 5.75 | 6.75 | 6.3 | 15.1 | 15.1 | 15.1 |
| | July 28 | 44 | 3.5 | 3.75 | 3.6 | 4.5 | 4.5 | 4.5 | 16 | 15.6 | 15.8 |
| | July 28 | 45 | 3 | 0 | 1.5 | 3.5 | 3.75 | 3.6 | 14.2 | 13.2 | 13.7 |
| | July 28 | 46 | 3 | 3 | 3.0 | 6 | 4 | 5.0 | 15.4 | 16.1 | 15.8 |
| | July 28 | 47 | 3.5 | 3.25 | 3.4 | 5.25 | 5.25 | 5.3 | 15.9 | 15.1 | 15.5 |
| | July 28 | 48 | 3.5 | 3.25 | 3.4 | 4.75 | 4.5 | 4.6 | 14.6 | 15.4 | 15.0 |
| | July 28 | 49 | 3.25 | 4 | 3.6 | 4.5 | 5.25 | 4.9 | 15 | 16.1 | 15.6 |
| | July 28 | 50 | 3 | 3 | 3.0 | 4.75 | 4.25 | 4.5 | 14.3 | 15.5 | 14.9 |
| | July 28 | 51 | 3.25 | 3.25 | 3.3 | 3.25 | 3.75 | 3.5 | 15.2 | 15.7 | 15.5 |
| | July 28 | 52 | 4 | 3.5 | 3.8 | 4.75 | 5.25 | 5.0 | 14.2 | 15.1 | 14.7 |
| | July 28 | 53 | 3 | 3.25 | 3.1 | 3 | 4 | 3.5 | 13.9 | 11.8 | 12.9 |
| | July 28 | 54 | 3 | 3 | 3.0 | 4.5 | 5 | 4.8 | 16.3 | 16 | 16.2 |
| | July 28 | 55 | 3.25 | 3 | 3.1 | 4.5 | 3.75 | 4.1 | 15.9 | 16.1 | 16.0 |
| | July 28 | 56 | 3.25 | 3 | 3.1 | 5 | 5.5 | 5.3 | 16.3 | 15.6 | 16.0 |
| | July 28 | 57 | 0 | 3 | 1.5 | 3.5 | 5 | 4.3 | 16 | 14.6 | 15.3 |
| | July 28 | 58 | 3.25 | 3.5 | 3.4 | 5 | 6 | 5.5 | 15 | 14.3 | 14.7 |
| Averages | | | | | 3.0 | | | 4.6 | | | 15.5 |

### Example 7

### Physiological and Morphological Descriptions

In accordance with one aspect of the present invention, there is provided a plant having the physiological and morphological characteristics of melon hybrid SVR03968106. A description of the physiological and morphological characteristics of such plants is presented in Table 7.

TABLE 7
Physiological and morphological characteristics of hybrid SVR03968106

| | | | Comparison |
|---|---|---|---|
| | | | Variety - |
| | CHARACTERISTIC | SVR03968106 | HALEY |
| 1. | Type | honey dew | honey dew |
| 2. | Area of best adaptation in the U.S.A. | most areas | most areas |
| 3. | Seedling | | |
| | length of hypocotyl (just before | long (Bimbo, | medium |
| | development of the first true leaf) | Ronda) | |
| | size of cotyledon | large (Bimbo, | medium |
| | | Nicolás) | |
| | intensity of green color of | medium (Candy, | medium |
| | cotyledon | Piel de Sapo) | |
| 4. | Leaf (mature blade of third leaf) | | |
| | shape | ovate | ovate |
| | lobes | shallowly lobed | shallowly lobed |
| | color | medium green | medium green |
| | RHS Color Chart value | 137A | 137A |
| | length | 103 mm | 95 mm |
| | width | 137 mm | 129 mm |
| | surface | pubescent | pubescent |
| 5. | Leaf Blade (fully developed but not old | | |
| | leaves, preferably between the 5th and | | |
| | 8th node when the plant has at least 11 | | |
| | nodes) | | |
| | size | medium (Candy, | medium |
| | | Total) | |
| | intensity of green color | medium (Doral, | medium |
| | | Galia) | |
| | development of lobes | medium (Piel de | medium |
| | | Sapol) | |
| | length of terminal lobe | medium (Clipper, | medium |
| | | Gama) | |
| | dentation of margin | medium (De | medium |
| | | Cavaillon espagnol, | |
| | | Piel de Sapo) | |
| | blistering | weak (Galia) | weak |
| 6. | Petiole | | |
| | attitude | semi-erect (Peko) | semi-erect |
| | length | medium (Arava, | long |
| | | Sancho) | |
| | time of male flowering | medium (Catagoria) | medium |
| | time of female flowering | medium (Braco, | early |
| | | Catagoria, Vital) | |
| 7. | Plant | | |
| | fertility - sex expression (at full | andro-monoecious | andro-monecious |
| | flowering) | (Piel de Saoi) | |
| | habit | vine | vine |
| 8. | Young fruit (green, unripe fruit before | | |
| | color change) | | |
| | hue of green color of skin | green (Lucas) | yellowish green |
| | intensity of green color of skin | light (Fimel) | light |
| | density of dots | dense (Arava) | dense |
| | size of dots | medium (Arava) | small |
| | contrast of dot color/ground color | medium (Arava) | medium |
| | conspicuousness of groove | absent or very weak | weak |
| | coloring | (Solarking) | |
| | intensity of groove coloring | light | light |
| | length of peduncle | short (Lince, Haros) | medium |
| | thickness of peduncle 1 cm from | medium (Geaprince, | medium |
| | fruit | Védrantais) | |
| | extension of darker area around | small (Boule d'or) | small |
| | peduncle | | |
| 9. | Fruit | | |
| | change of skin color from young | early in fruit | late in fruit |
| | fruit to maturity | development | development |
| | | (Alpha, Charantais, | |
| | | Clipper) | |
| | length | long (Catagoria, | long |
| | | Toledo) | |
| | length (at edible maturity) | 17 cm | 18 cm |
| | diameter | broad (Albino, | medium |
| | | Kinka) | |
| | diameter (at edible maturity) | 13 cm | 16 cm |
| | ratio length/diameter | medium (Doral, | medium |
| | | Tendral Negro) | |
| | weight (at edible maturity) | 3250 gm | 2435 gm |
| | position of maximum diameter | at middle (Piel de | at middle |
| | | Sapo, Védrantais) | |
| | shape in longitudinal section | broad elliptic | broad elliptic |
| | | (Corin, Sardo) | |
| | surface (at edible maturity) | smooth | smooth |
| | blossom scar (at edible maturity) | obscure | conspicuous |
| | rib presence (at edible maturity) | absent | absent |
| | shipping quality (at edible | poor (Home | excellent |
| | maturity) | Garden) | |
| | abscission (at edible maturity) | when ripe | do not abscise |
| | maturity (number of days from | 108 | 109 |
| | seeding to harvest) | | |
| | ground color of skin | yellow (Amarillo- | green |
| | | Canario, Edén, | |
| | | Galia, Passport, | |
| | | Solarking) | |
| | intensity of ground color of skin | medium | light |
| | hue of ground color of skin | yellowish | yellowish |
| | | (Geaprince, | |
| | | Supporter) | |
| | density of dots | medium (Petit Gris | very dense |
| | | de Rennes) | |
| | size of dots | medium (Toledo) | medium |
| | color of dots | white (Edén) | white |
| | intensity of color of dots | light (Kinka, Mesol) | light |
| | density of patches | absent or very | absent or very |
| | | sparse (Rochet) | sparse |
| | warts | absent (Piel de | absent |
| | | Sapo) | |
| | strength of attachment of peduncle | very weak (Edén) | strong |
| | at maturity | | |
| | shape of base | truncate (Zatta) | rounded |
| | shape of apex | rounded (Alpha, | rounded |
| | | Honey Dew) | |
| | size of pistil scar | large (Drake, | large |
| | | Supermarket) | |
| | grooves | absent or very | absent or very |
| | | weakly expressed | weakly expressed |
| | | (Piel de Sapo, | |
| | | Arava) | |
| | creasing of surface | absent or very weak | absent or very |
| | | (Védrantais) | weak |
| | cork formation | absent (Alpha) | absent |
| | rate of change of skin color from | slow (Goloso) | medium |
| | maturity to over maturity | | |
| | width of flesh in longitudinal | thick (Tito) | medium |
| | section (at position of maximum | | |
| | fruit diameter) | | |
| | main color of flesh | greenish white | greenish white |
| | | (Galia) | |
| | secondary salmon coloring of flesh | absent or very weak | absent or very |
| | | (Gustal) | weak |
| | firmness of flesh | firm (Braco, | medium |
| | | Geamar) | |
| | at over maturity: hue of color of | creamish (Figaro, | creamish |
| | skin | Vendôme) | |
| | time of ripening | very early | early |
| | | (Goldstar, Sun) | |
| | shelf life of fruit | medium (Clipper) | medium |
| 10. | Flesh | | |
| | color near cavity (at edible | yellowish-white | creamy-green |
| | maturity) | | |
| | RHS Color Chart value | 158A | 155A |
| | color in center (at edible maturity) | yellowish-cream | green |
| | RHS Color Chart value | 160C | 141C |
| | color near rind (at edible maturity) | yellowish-white | creamy-green |
| | RHS Color Chart value | 158B | 155A |
| | refractometer % soluable solids | 10% | 14% |
| | (center of flesh) | | |
| | aroma (at edible maturity) | strong | strong |
| | flavor (at edible maturity) | very spicy | somewhat spicy |
| 11. | Seed Cavity | | |
| | length | 123 mm | 130 mm |
| | width | 86 mm | 73 mm |
| | shape in cross section | triangular | circular |
| 12. | Seed (fully developed and dry seeds, | | |
| | after washing and drying in the shade) | | |
| | length | very long (Albino) | medium |
| | width | medium (Avara, | narrow |
| | | Sancho) | |
| | shape | not pine-nut shape | not pine-nut |
| | | (Toledo) | shape |
| | color | cream yellow | cream yellow |
| | | (Galia, Piel de | |
| | | Sapo) | |
| | intensity of color | dark (Doral) | medium |
| | number of seeds per fruit | 527 | 583 |
| | grams per 1,000 seeds | 39 gm | 42 gm |
| 13. | Rind | | |
| | net presence | absent | absent |
| | texture: soft, firm or hard | soft | hard |
| | thickness at medial | 2 mm | 3 mm |
| | primary color (at edible maturity) | cream | buff |
| | RHS color chart value | 157A | 157A |
| | mottling color (at edible maturity) | whitish-yellow | buff |
| | RHS color chart value | 158B | 157D |
| | primary color (at full maturity) | cream | buff |
| | RHS color chart value | 157A | 157A |
| | mottling color (at full maturity) | whitish-yellow | buff |
| | RHS color chart value | 158B | 157D |

*These are typical values. Values may vary due to environment. Other values that are substantially equivalent are also within the scope of the invention.

In accordance with another aspect of the present invention, there is provided a plant having the physiological and morphological characteristics of melon hybrid SVR03968074. A description of the physiological and morphological characteristics of such plants is presented in Table 8.

TABLE 8
Physiological and morphological characteristics of hybrid SVR03968074

| | | | Comparison |
|---|---|---|---|
| | | | Variety |
| | CHARACTERISTIC | SVR03968074 | TAM DEW IMP |
| 1. | Type | honey dew | honey dew |
| 2. | Area of best adaptation in the U.S.A. | most areas | most areas |
| 3. | Seedling | | |
| | length of hypocotyl | medium (Doral, | long |
| | | Futuro) | |
| | size of cotyledon | medium (Futuro, | large |
| | | Sancho) | |
| | intensity of green color of | medium (Candy, | medium |
| | cotyledon | Piel de Sapo) | |
| 4. | Leaf (mature blade of third leaf) | | |
| | shape | ovate | ovate |
| | lobes | shallowly lobed | shallowly lobed |
| | color | medium green | medium green |
| | RHS Color Chart value | 136A | 137A |
| | length | 107 mm | 96 mm |
| | width | 269 mm | 126 mm |
| | surface | pubescent | pubescent |
| 5. | Leaf Blade (fully developed but not old | | |
| | leaves, preferably between the 5th and | | |
| | 8th node when the plant has at least 11 | | |
| | nodes) | | |
| | size | medium (Candy, | medium |
| | | Total) | |
| | intensity of green color | medium (Doral, | medium |
| | | Galia) | |
| | development of lobes | medium (Piel de | weak |
| | | Sapol) | |
| | length of terminal lobe | medium (Clipper, | medium |
| | | Gama) | |
| | dentation of margin | medium (De | medium |
| | | Cavaillon espagnol, | |
| | | Piel de Sapo) | |
| | blistering | weak (Galia) | weak |
| 6. | Petiole | | |
| | attitude | semi-erect (Peko) | semi-erect |
| | length | medium (Arava, | medium |
| | | Sancho) | |
| 7. | Plant | | |
| | fertility - sex expression (at full | andro-monoecious | monecious |
| | flowering) | (Piel de Saoi) | |
| | habit | vine | vine |
| 8. | Young fruit (green, unripe fruit before | | |
| | color change) | | |
| | hue of green color of skin | yellowish green | yellowish green |
| | | (Fimel) | |
| | intensity of green color of skin | light (Fimel) | light |
| | density of dots | medium (Lucss) | very dense |
| | size of dots | medium (Arava) | small |
| | contrast of dot color/ground color | medium (Arava) | medium |
| | conspicuousness of groove | absent or very weak | weak |
| | coloring | (Solarking) | |
| | intensity of groove coloring | light | light |
| | length of peduncle | medium (Arava, | long |
| | | Romeo) | |
| | thickness of peduncle 1 cm from | medium (Geaprince, | medium |
| | fruit | Védrantais) | |
| | extension of darker area around | absent or very small | medium |
| | peduncle | (Doral) | |
| 9. | Fruit | | |
| | change of skin color from young | late in fruit | late in fruit |
| | fruit to maturity | development | development |
| | | (Amarillo Oro, | |
| | | Galia) | |
| | length | medium (Marina, | medium |
| | | Spanglia) | |
| | length (at edible maturity) | 16 cm | 17 cm |
| | diameter | medium (Catagoria, | medium |
| | | Galia) | |
| | diameter (at edible maturity) | 11 cm | 15 cm |
| | ratio length/diameter | small to medium | very small to |
| | | (Aril, Edén) | small |
| | weight (at edible maturity) | 2371 gm | 1942 gm |
| | position of maximum diameter | at middle (Piel de | at middle |
| | | Sapo, Védrantais) | |
| | shape in longitudinal section | broad elliptic | broad elliptic |
| | | (Corin, Sardo) | |
| | surface (at edible maturity) | smooth | warted |
| | blossom scar (at edible maturity) | conspicuous | obscure |
| | rib presence (at edible maturity) | absent | absent |
| | shipping quality (at edible | good to excellent | excellent |
| | maturity) | | |
| | abscission (at edible maturity) | when ripe | do not abscise |
| | maturity (number of days from | 108 | 108 |
| | seeding to harvest) | | |
| | ground color of skin | yellow (Amarillo- | green |
| | | Canario, Edén, | |
| | | Galia, Passport, | |
| | | Solarking) | |
| | intensity of ground color of skin | light | light |
| | hue of ground color of skin | yellowish | whitish |
| | | (Geaprince, | |
| | | Supporter) | |
| | density of dots | dense (Piel de Sapo) | dense |
| | size of dots | small (Doral) | small |
| | color of dots | white (Edén) | white |
| | intensity of color of dots | light (Kinka, Mesol) | light |
| | density of patches | absent or very | absent or very |
| | | sparse (Rochet) | sparse |
| | warts | absent (Piel de | absent |
| | | Sapo) | |
| | strength of attachment of peduncle | very weak (Edén) | very strong |
| | at maturity | | |
| | shape of base | rounded (Arava) | pointed |
| | shape of apex | rounded (Alpha, | rounded |
| | | Honey Dew) | |
| | size of pistil scar | large (Drake, | medium |
| | | Supermarket) | |
| | grooves | absent or very | absent or very |
| | | weakly expressed | weakly expressed |
| | | (Piel de Sapo, | |
| | | Arava) | |
| | creasing of surface | weak (Melchor, | weak |
| | | Sirocco) | |
| | cork formation | absent (Alpha) | absent |
| | rate of change of skin color from | absent or very slow | absent or very |
| | maturity to over maturity | (Clipper, Doral, | slow |
| | | Galia, Honey Dew, | |
| | | Piel de Sapo) | |
| | width of flesh in longitudinal | medium (Toledo) | medium |
| | section (at position of maximum | | |
| | fruit diameter) | | |
| | main color of flesh | greenish white | greenish white |
| | | (Galia) | |
| | secondary salmon coloring of flesh | absent or very weak | absent or very |
| | | (Gustal) | weak |
| | firmness of flesh | soft (Galia, Marina) | firm |
| | at over maturity: hue of color of | orangish yellow | |
| | skin | (Drake, Gama) | |
| | at over maturity: intensity of | medium (Futuro) | |
| | yellow color of skin | | |
| 10. | Flesh | | |
| | color near cavity (at edible | white-yellow | white-green |
| | maturity) | | |
| | RHS Color Chart value | 158C | 143A |
| | color in center (at edible maturity) | yellow-green | white-green |
| | RHS Color Chart value | 144C | 143A |
| | color near rind (at edible maturity) | white-yellow | white-cream |
| | RHS Color Chart value | 158A | 157A |
| | refractometer % soluable solids | 16% | 12% |
| | (center of flesh) | | |
| | aroma (at edible maturity) | faint | faint |
| | flavor (at edible maturity) | somewhat spicy | mild |
| 11. | Seed Cavity | | |
| | length | 108 mm | 118 mm |
| | width | 62 mm | 75 mm |
| | shape in cross section | circular | circular |
| 12. | Seed (fully developed and dry seeds, | | |
| | after washing and drying in the shade) | | |
| | length | medium (Avara, | medium |
| | | Sancho) | |
| | width | medium (Avara, | narrow |
| | | Sancho) | |
| | shape | pine-nut shape (Piel | pine-nut shape |
| | | de Sapo) | |
| | color | cream yellow | cream yellow |
| | | (Galia, Piel de | |
| | | Sapo) | |
| | intensity of color | medium (Galia) | medium |
| | time of male flowering | medium (Catagoria) | late |
| | time of female flowering | medium (Braco, | late |
| | | Catagoria, Vital) | |
| | time of ripening | very early | early |
| | | (Goldstar, Sun) | |
| | shelf life of fruit | medium (Clipper) | short |
| | number of seeds per fruit | 415 | 528 |
| | grams per 1,000 seeds | 33 gm | 36 gm |
| 13. | Rind | | |
| | net presence | absent | absent |
| | texture: soft, firm or hard | soft | hard |
| | thickness at medial | 3 mm | 4 mm |
| | primary color (at edible maturity) | white-yellow | cream |
| | RHS color chart value | 158B | 157B |
| | mottling color (at edible maturity) | gray-white | cream |
| | RHS color chart value | 196B | 157C |
| | primary color (at full maturity) | white-yellow | cream |
| | RHS color chart value | 158B | 157B |
| | mottling color (at full maturity) | gray-white | cream |
| | RHS color chart value | 196B | 157C |

*These are typical values. Values may vary due to environment. Other values that are substantially equivalent are also within the scope of the invention.

In accordance with another aspect of the present invention, there is provided a plant having the physiological and morphological characteristics of melon line HDG39-2021AN. A description of the physiological and morphological characteristics of such plants is presented in Table 9.

TABLE 9
Physiological and Morphological Characteristics of Line HDG39-2021AN

| | | | Comparison |
|---|---|---|---|
| | | | Variety |
| | CHARACTERISTIC | HDG39-2021AN | EMERALD |
| 1. | Type | honey dew | honey dew |
| 2. | Area of best adaptation in the U.S.A. | most areas | most areas |
| 3. | Seedling | | |
| | length of hypocotyl (just before | very long (Noy) | long |
| | development of first true leaf) | | |
| | size of cotyledon | very large (Noy) | large |
| | intensity of green color of | medium (Candy, Piel | light |
| | cotyledon | de Sapo) | |
| 4. | Leaf (mature blade of third leaf) | | |
| | shape | ovate | reniform |
| | lobes | shallowly lobed | shallowly lobed |
| | color | dark green (Rio | medium green |
| | | Gold) | |
| | RHS Color Chart value | 136A | 147A |
| | length | 113 mm | 81 mm |
| | width | 156 mm | 107 mm |
| | surface | pubescent | pubescent |
| 5. | Leaf Blade (fully developed but not old | | |
| | leaves, preferably between the 5th and | | |
| | 8th node when the plant has at least 11 | | |
| | nodes) | | |
| | size | large (Don, Sucrero) | medium |
| | intensity of green color | dark (Gama, Gustal) | medium |
| | development of lobes | medium (Piel de | medium |
| | | Sapol) | |
| | length of terminal lobe | medium (Clipper, | medium |
| | | Gama) | |
| | dentation of margin | medium (De | medium |
| | | Cavaillon espagnol, | |
| | | Piel de Sapo) | |
| | blistering | weak (Galia) | medium |
| 6. | Petiole | | |
| | attitude | semi-erect (Peko) | erect |
| | length | long (Goldgen) | long |
| 7. | Plant | | |
| | fertility - sex expression (at full | andro-monoecious | andro-monecious |
| | flowering) | (Piel de Saoi) | |
| | habit | vine | vine |
| | time of male flowering | late (Nicolás, Rocin) | medium |
| | time of female flowering | medium (Braco, | medium |
| | | Catagoria, Vital) | |
| 8. | Young fruit (green unripe fruit before | | |
| | color change) | | |
| | hue of green color of skin | yellowish green | green |
| | | (Fimel) | |
| | intensity of green color of skin | light (Fimel) | light |
| | density of dots | medium (Lucss) | dense |
| | size of dots | medium (Arava) | small |
| | contrast of dot color/ground color | weak (Lucas) | weak |
| | conspicuousness of groove coloring | weak (Geaprince, | absent or very |
| | | Total) | weak |
| | intensity of groove coloring | light | |
| | length of peduncle | medium (Arava, | medium |
| | | Romeo) | |
| | thickness of peduncle 1 cm from | medium (Geaprince, | medium |
| | fruit | Védrantais) | |
| | extension of darker area around | absent or very small | absent or very |
| | peduncle | (Doral) | small |
| 9. | Fruit | | |
| | change of skin color from young | late in fruit | late in fruit |
| | fruit to maturity | development | development |
| | | (Amarillo Oro, | |
| | | Galia) | |
| | length | medium (Marina, | very long |
| | | Spanglia) | |
| | length (at edible maturity) | 16 cm | 20 cm |
| | diameter | broad (Albino, | medium |
| | | Kinka) | |
| | diameter (at edible maturity) | 18 cm | 14 cm |
| | ratio length/diameter | medium to large | very large |
| | | (Sirocco, Verdol) | |
| | weight (at edible maturity) | 2786 gm | 2275 gm |
| | position of maximum diameter | at middle (Piel de | at middle |
| | | Sapo, Védrantais) | |
| | shape in longitudinal section | broad elliptic (Corin, | medium elliptic/ |
| | | Sardo) | elongate- |
| | | | cylindrical |
| | surface (at edible maturity) | smooth | corrugated |
| | blossom scar (at edible maturity) | conspicuous | conspicuous |
| | rib presence (at edible maturity) | absent | absent |
| | shipping quality (at edible maturity) | excellent (Long | excellent |
| | | Distance Shipping) | |
| | abscission (at edible maturity) | when overripe | do not abscise |
| | maturity (number of days from | 108 | 107 |
| | seeding to harvest) | | |
| | ground color of skin | yellow (Amarillo- | green |
| | | Canario, Edén, Galia, | |
| | | Passport, Solarking) | |
| | intensity of ground color of skin | medium | light |
| | hue of ground color of skin | yellowish | whitish |
| | | (Geaprince, | |
| | | Supporter) | |
| | density of dots | dense (Piel de Sapo) | dense |
| | size of dots | small (Doral) | small |
| | color of dots | white (Edén) | white |
| | intensity of color of dots | light (Kinka, Mesol) | medium |
| | density of patches | absent or very sparse | absent or very |
| | | (Rochet) | sparse |
| | warts | absent (Piel de Sapo) | absent |
| | strength of attachment of peduncle | very weak (Edén) | strong |
| | at maturity | | |
| | shape of base | rounded (Arava) | pointed |
| | shape of apex | rounded (Alpha, | pointed |
| | | Honey Dew) | |
| | size of pistil scar | large (Drake, | medium |
| | | Supermarket) | |
| | grooves | absent or very | absent or very |
| | | weakly expressed | weakly |
| | | (Piel de Sapo, Arava) | expressed |
| | color of grooves | | green |
| | creasing of surface | absent or very weak | absent or very |
| | | (Védrantais) | weak |
| | cork formation | absent (Alpha) | absent |
| | rate of change of skin color from | medium (Futuro, | absent or very |
| | maturity to over maturity | Vendôme, Dulcinea) | slow |
| | width of flesh in longitudinal | thick (Tito) | medium |
| | section (at position of maximum | | |
| | fruit diameter) | | |
| | main color of flesh | greenish white | green |
| | | (Galia) | |
| | secondary salmon coloring of flesh | absent or very weak | absent or very |
| | | (Gustal) | weak |
| | firmness of flesh (in central third of | soft (Galia, Marina) | firm |
| | fruit) | | |
| | at over maturity: hue of color of | yellow (Futuro, | |
| | skin | Marina) | |
| | at over maturity: intensity of yellow | light (Dulcinea) | |
| | color of skin | | |
| | time of ripening | early (Galia) | early |
| | shelf life of fruit | long (Piel de Sapo) | long |
| 10. | Flesh | | |
| | color near cavity (at edible | whitish-cream | green |
| | maturity) | | |
| | RHS Color Chart value | 155C | 142B |
| | color in center (at edible maturity) | whitish-cream | greenish-cream |
| | RHS Color Chart value | 160B | 142D |
| | color near rind (at edible maturity) | green | greenish-cream |
| | RHS Color Chart value | 143B | 143C |
| | refractometer % soluable solids | 11% | 27% |
| | (center of flesh) | | |
| | aroma (at edible maturity) | strong | faint |
| | flavor (at edible maturity) | somewhat spicy | mild |
| 11. | Seed Cavity | | |
| | length | 109 mm | 130 mm |
| | width | 87 mm | 81 mm |
| | shape in cross section | circular | circular |
| 12. | Seed (fully developed and dry seeds, | | |
| | after washing and drying in the shade) | | |
| | length | long (Amarillo Oro, | long |
| | | Toledo) | |
| | width | medium (Avara, | medium |
| | | Sancho) | |
| | shape | not pine-nut shape | not pine-nut |
| | | (Toledo) | shape |
| | color | cream yellow (Galia, | cream yellow |
| | | Piel de Sapo) | |
| | intensity of color | medium (Galia) | medium |
| | number of seeds per fruit | 522 | 526 |
| | grams per 1,000 seeds | 39 gm | 38 gm |
| 13. | Rind | | |
| | net presence | absent | absent |
| | texture (soft, firm or hard) | firm | hard |
| | thickness at medial | 3 mm | 3 mm |
| | primary color (at edible maturity) | whitish-buff | whitish-cream |
| | RHS color chart value | 163D | 157A |
| | mottling color (at edible maturity) | whitish-buff | whitish-cream |
| | RHS color chart value | 157B | 157B |
| | primary color (at full maturity) | whitish-buff | greenish-white |
| | RHS color chart value | 163D | 145D |
| | mottling color (at full maturity) | whitish-buff | whitish-buff |
| | RHS color chart value | 157B | 158C |

*These are typical values. Values may vary due to environment. Other values that are substantially equivalent are also within the scope of the invention.

In accordance with another aspect of the present invention, there is provided a plant having the physiological and morphological characteristics of melon line HDG39-2023AN. A description of the physiological and morphological characteristics of such plants is presented in Table 10.

TABLE 10
Physiological and Morphological Characteristics of Line HDG39-2023AN

| | | | Comparison |
|---|---|---|---|
| | | | Variety |
| | | | TAM DEW |
| | CHARACTERISTIC | HDG39-2023AN | IMP |
| 1. | Type | honey dew | honey dew |
| 2. | Area of best adaptation in the U.S.A. | most areas | most areas |
| 3. | Seedling | | |
| | length of hypocotyl (just before | very short (Golden | long |
| | development of first true leaf) | Crispy) | |
| | size of cotyledon | small (Candy, | large |
| | | Lunasol) | |
| | intensity of green color of | medium (Candy, Piel | medium |
| | cotyledon | de Sapo) | |
| 4. | Leaf (mature blade of third leaf) | | |
| | shape | ovate | ovate |
| | lobes | shallowly lobed | shallowly lobed |
| | color | medium green | medium green |
| | RHS Color Chart value | 137A | 137A |
| | length | 96 mm | 96 mm |
| | width | 126 mm | 126 mm |
| | surface | pubescent | pubescent |
| 5. | Leaf Blade (fully developed but not old | | |
| | leaves, preferably between the 5^{th} and | | |
| | 8^{th} node when the plant has at least 11 | | |
| | nodes) | | |
| | size | medium (Candy, | medium |
| | | Total) | |
| | intensity of green color | medium (Doral, Galia) | medium |
| | development of lobes | medium (Piel de | weak |
| | | Sapol) | |
| | length of terminal lobe | long (Gustal, Primal) | medium |
| | dentation of margin | medium (De | medium |
| | | Cavaillon espagnol, | |
| | | Piel de Sapo) | |
| | blistering | medium (Costa) | weak |
| 6. | Petiole | | |
| | attitude | semi-erect (Peko) | semi-erect |
| | length | medium (Arava, | medium |
| | | Sancho) | |
| 7. | Plant | | |
| | fertility - sex expression (at full | andro-monoecious | monecious |
| | flowering) | (Piel de Saoi) | |
| | habit | vine | vine |
| | time of male flowering | medium (Catagoria) | late |
| | time of female flowering | medium (Braco, | late |
| | | Catagoria, Vital) | |
| 8. | Young fruit (green unripe fruit before | | |
| | color change) | | |
| | hue of green color of skin | whitish green (Geasol) | yellowish green |
| | intensity of green color of skin | light (Fimel) | light |
| | density of dots | dense (Arava) | very dense |
| | size of dots | medium (Arava) | small |
| | contrast of dot color/ground color | weak (Lucas) | medium |
| | conspicuousness of groove coloring | weak (Geaprince, | weak |
| | | Total) | |
| | intensity of groove coloring | medium (Gama, | light |
| | | Topper) | |
| | length of peduncle | medium (Arava, | long |
| | | Romeo) | |
| | thickness of peduncle 1 cm from | medium (Geaprince, | medium |
| | fruit | Védrantais) | |
| | extension of darker area around | medium (Mirasol | medium |
| | peduncle | Geaprince) | |
| 9. | Fruit | | |
| | change of skin color from young | late in fruit | late in fruit |
| | fruit to maturity | development | development |
| | | (Amarillo Oro, Galia) | |
| | length | medium (Marina, | medium |
| | | Spanglia) | |
| | length (at edible maturity) | 17 cm | 17 cm |
| | diameter | medium (Catagoria, | medium |
| | | Galia) | |
| | diameter (at edible maturity) | 11 cm | 15 cm |
| | ratio length/diameter | small to medium | very small to |
| | | (Aril, Edén) | small |
| | weight (at edible maturity) | 2031 gm | 1942 gm |
| | position of maximum diameter | at middle (Piel de | at middle |
| | | Sapo, Védrantais) | |
| | shape in longitudinal section | broad elliptic (Corin, | broad elliptic |
| | | Sardo) | |
| | surface (at edible maturity) | smooth | warted |
| | blossom scar (at edible maturity) | conspicuous | obscure |
| | rib presence (at edible maturity) | absent | absent |
| | shipping quality (at edible maturity) | poor (Home Garden) | excellent |
| | abscission (at edible maturity) | when ripe | do not abscise |
| | maturity (number of days from | 108 | 108 |
| | seeding to harvest | | |
| | ground color of skin | yellow (Amarillo- | green |
| | | Canario, Edén, Galia, | |
| | | Passport, Solarking) | |
| | intensity of ground color of skin | light | light |
| | hue of ground color of skin | yellowish (Geaprince, | whitish |
| | | Supporter) | |
| | density of dots | dense (Piel de Sapo) | dense |
| | size of dots | small (Doral) | small |
| | color of dots | white (Edén) | white |
| | intensity of color of dots | light (Kinka, Mesol) | light |
| | density of patches | absent or very sparse | absent or very |
| | | (Rochet) | sparse |
| | warts | absent (Piel de Sapo) | absent |
| | strength of attachment of peduncle | weak (Arava, | very strong |
| | at maturity | Maestro) | |
| | shape of base | rounded (Arava) | pointed |
| | shape of apex | rounded (Alpha, | rounded |
| | | Honey Dew) | |
| | size of pistil scar | medium (Chartenais, | medium |
| | | Eros, Verdol) | |
| | grooves | absent or very weakly | absent or very |
| | | expressed (Piel de | weakly |
| | | Sapo, Arava) | expressed |
| | creasing of surface | absent or very weak | weak |
| | | (Védrantais) | |
| | cork formation | absent (Alpha) | absent |
| | rate of change of skin color from | absent or very slow | absent or very |
| | maturity to over maturity | (Clipper, Doral, Galia, | slow |
| | | Honey Dew, Piel de | |
| | | Sapo) | |
| | width of flesh in longitudinal | thick (Tito) | medium |
| | section (at position of maximum | | |
| | fruit diameter) | | |
| | main color of flesh | greenish white (Galia) | greenish white |
| | secondary salmon coloring of flesh | absent or very weak | absent or very |
| | | (Gustal) | weak |
| | firmness of flesh (in central third of | medium (Sancho, | firm |
| | fruit) | Supporter) | |
| | at over maturity: hue of color of | yellow (Futuro, | |
| | skin | Marina) | |
| | at over maturity: intensity of yellow | medium (Futuro) | |
| | color of skin | | |
| | time of ripening | very early (Goldstar, | early |
| | | Sun) | |
| | shelf life of fruit | medium (Clipper) | short |
| 10. | Flesh | | |
| | color near cavity (at edible | white-green | white-green |
| | maturity) | | |
| | RHS Color Chart value | 157A | 143A |
| | color in center (at edible maturity) | yellow-green | white-green |
| | RHS Color Chart value | 145A | 143A |
| | color near rind (at edible maturity) | creamy-yellow | white-cream |
| | RHS Color Chart value | 162D | 157A |
| | refractometer % soluble solids | 15% | 12% |
| | (center of flesh) | | |
| | aroma (at edible maturity) | strong | faint |
| | flavor (at edible maturity) | very spicy | mild |
| 11. | Seed Cavity | | |
| | length | 63 mm | 118 mm |
| | width | 36 mm | 75 mm |
| | shape in cross section | circular | circular |
| 12. | Seed (fully developed and dry seeds, | | |
| | after washing and drying in the shade) | | |
| | length | medium (Avara, | medium |
| | | Sancho) | |
| | width | medium (Avara, | narrow |
| | | Sancho) | |
| | shape | not pine-nut shape | pine-nut shape |
| | | (Toledo) | |
| | color | cream yellow (Galia, | cream yellow |
| | | Piel de Sapo) | |
| | intensity of color | light (Goldgen) | medium |
| | number of seeds per fruit | 519 | 528 |
| | grams per 1,000 seeds | 31 gm | 36 gm |
| 13. | Rind | | |
| | net presence | absent | absent |
| | texture (soft, firm or hard) | firm | hard |
| | thickness at medial | 3 mm | 4 mm |
| | primary color (at edible maturity) | white-yellow | cream |
| | RHS color chart value | 195A | 157B |
| | mottling color (at edible maturity) | white-yellow | cream |
| | RHS color chart value | 158B | 157C |
| | primary color (at full maturity) | white-yellow | cream |
| | RHS color chart value | 195A | 157B |
| | mottling color (at full maturity) | white-yellow | cream |
| | RHS color chart value | 158B | 157C |

*These are typical values. Values may vary due to environment. Other values that are substantially equivalent are also within the scope of the invention.

In accordance with another aspect of the present invention, there is provided a plant having the physiological and morphological characteristics of melon line HDG39-2022AN. A description of the physiological and morphological characteristics of such plants is presented in Table 11.

TABLE 11
Physiological and Morphological Characteristics of Line HDG39-2022AN

| | | | Comparison |
|---|---|---|---|
| | | | Variety |
| | CHARACTERISTIC | HDG 39-2022 AN | TAM DEW IMP |
| 1. | Type | honey dew | honey dew |
| 2. | Area of best adaptation in the U.S.A. | most areas | most areas |
| 3. | Seedling | | |
| | length of hypocotyl | very long (Noy) | long |
| | size of cotyledon | medium (Futuro, | large |
| | | Sancho) | |
| | intensity of green color of | medium (Candy, | medium |
| | cotyledon | Piel de Sapo) | |
| 4. | Leaf (mature blade of third leaf) | | |
| | shape | ovate | ovate |
| | lobes | shallowly lobed | shallowly lobed |
| | color | medium green | medium green |
| | RHS Color Chart value | 137A | 137A |
| | length | 107 mm | 96 mm |
| | width | 134 mm | 126 mm |
| | surface | pubescent | pubescent |
| 5. | Leaf Blade (fully developed but not old | | |
| | leaves, preferably between the 5^{th} and | | |
| | 8^{th} node when the plant has at least 11 | | |
| | nodes) | | |
| | size | medium (Candy, | medium |
| | | Total) | |
| | intensity of green color | light (Fimel, Yuma) | medium |
| | development of lobes | medium (Piel de | weak |
| | | Sapol) | |
| | length of terminal lobe | medium (Clipper, | medium |
| | | Gama) | |
| | dentation of margin | medium (De | medium |
| | | Cavaillon espagnol, | |
| | | Piel de Sapo) | |
| | blistering | weak (Galia) | weak |
| 6. | Petiole | | |
| | attitude | semi-erect (Peko) | semi-erect |
| | length | medium (Arava, | medium |
| | | Sancho) | |
| 7. | Plant | | |
| | fertility - sex expression (at full | andro-monoecious | monecious |
| | flowering) | (Piel de Saoi) | |
| | habit | vine | vine |
| 8. | Young fruit (green, unripe fruit before | | |
| | color change) | | |
| | hue of green color of skin | yellowish green | yellowish green |
| | | (Fimel) | |
| | intensity of green color of skin | very light | light |
| | | (Solarking) | |
| | density of dots | dense (Arava) | very dense |
| | size of dots | medium (Arava) | small |
| | contrast of dot color/ground color | weak (Lucas) | medium |
| | conspicuousness of groove | medium (Gama) | weak |
| | coloring | | |
| | intensity of groove coloring | medium (Gama, | light |
| | | Topper) | |
| | length of peduncle | short (Lince, Haros) | long |
| | thickness of peduncle 1 cm from | thick (Chartenais, | medium |
| | fruit | Doral) | |
| | extension of darker area around | absent or very small | medium |
| | peduncle | (Doral) | |
| 9. | Fruit | | |
| | change of skin color from young | late in fruit | late in fruit |
| | fruit to maturity | development | development |
| | | (Amarillo Oro, | |
| | | Galia) | |
| | length | medium (Marina, | medium |
| | | Spanglia) | |
| | length (at edible maturity) | 15 cm | 17 cm |
| | diameter | broad (Albino, | medium |
| | | Kinka) | |
| | diameter (at edible maturity) | 11 cm | 15 cm |
| | ratio length/diameter | small to medium | very small to |
| | | (Aril, Edén) | small |
| | weight (at edible maturity) | 2361 gm | 1942 gm |
| | position of maximum diameter | at middle (Piel de | at middle |
| | | Sapo, Védrantais) | |
| | shape in longitudinal section | broad elliptic | broad elliptic |
| | | (Corin, Sardo) | |
| | surface (at edible maturity) | smooth | warted |
| | blossom scar (at edible maturity) | conspicuous | obscure |
| | rib presence (at edible maturity) | absent | absent |
| | shipping quality (at edible | poor (Home | excellent |
| | maturity) | Garden) | |
| | abscission (at edible maturity) | when ripe | do not abscise |
| | maturity (number of days from | 108 | 108 |
| | seeding to harvest) | | |
| | ground color of skin | yellow (Amarillo- | green |
| | | Canario, Edén, | |
| | | Galia, Passport, | |
| | | Solarking) | |
| | intensity of ground color of skin | medium | light |
| | hue of ground color of skin | yellowish | whitish |
| | | (Geaprince, | |
| | | Supporter) | |
| | density of dots | medium (Petit Gris | dense |
| | | de Rennes) | |
| | size of dots | small (Doral) | small |
| | color of dots | yellow (Piel de | white |
| | | Sapo) | |
| | intensity of color of dots | light (Kinka, | light |
| | | Mesol) | |
| | density of patches | | absent or very |
| | | | sparse |
| | warts | absent (Piel de | absent |
| | | Sapo) | |
| | strength of attachment of peduncle | strong (Clipper, | very strong |
| | at maturity | Costa) | |
| | shape of base | rounded (Arava) | pointed |
| | shape of apex | rounded (Alpha, | rounded |
| | | Honey Dew) | |
| | size of pistil scar | large (Drake, | medium |
| | | Supermarket) | |
| | grooves | absent or very | absent or very |
| | | weakly expressed | weakly expressed |
| | | (Piel de Sapo, | |
| | | Arava) | |
| | creasing of surface | absent or very weak | weak |
| | | (Védrantais) | |
| | cork formation | absent (Alpha) | absent |
| | rate of change of skin color from | fast (Corin, Marina, | absent or very |
| | maturity to over maturity | Nembo) | slow |
| | width of flesh in longitudinal | thick (Tito) | medium |
| | section (at position of maximum | | |
| | fruit diameter) | | |
| | main color of flesh | yellowish white | greenish white |
| | | (Guarani) | |
| | secondary salmon coloring of flesh | weak (Floraprince, | absent or very |
| | | Toledo) | weak |
| | firmness of flesh (in central third of | soft (Galia, Marina) | firm |
| | the fruit) | | |
| | at over maturity: hue of color of | yellow (Futuro, | |
| | skin | Marina) | |
| | at over maturity: intensity of | light (Dulcinea) | |
| | yellow color of skin | | |
| 10. | Flesh | | |
| | color near cavity (at edible | white-cream | white-green |
| | maturity) | | |
| | RHS color chart value | 155B | 143A |
| | color in center (at edible maturity) | green-white | white-green |
| | RHS color chart value | 144D | 143A |
| | color near rind (at edible maturity) | white-cream | white-cream |
| | RHS color chart value | 158A | 157A |
| | refractometer % soluable solids | 15% | 12% |
| | (center of flesh) | | |
| | aroma (at edible maturity) | strong | faint |
| | flavor (at edible maturity) | very spicy | mild |
| 11. | Seed Cavity | | |
| | length | 101 mm | 118 mm |
| | width | 65 mm | 75 mm |
| | shape in cross section | triangular | circular |
| 12. | Seed (fully developed and dry seeds, | | |
| | after washing and drying in the shade) | | |
| | length | long (Amarillo Oro, | medium |
| | | Toledo) | |
| | width | broad (Amarillo | narrow |
| | | Oro) | |
| | shape | pine-nut shape (Piel | pine-nut shape |
| | | de Sapo) | |
| | color | cream yellow | cream yellow |
| | | (Galia, Piel de | |
| | | Sapo) | |
| | intensity of color | medium (Galia) | medium |
| | time of male flowering | medium (Catagoria) | late |
| | time of female flowering | early (Clipper) | late |
| | time of ripening | early (Galia) | early |
| | shelf life of fruit | medium (Clipper) | short |
| | number of seeds per fruit | 452 | 528 |
| | grams per 1,000 seeds | 36 gm | 36 gm |
| 13. | Rind | | |
| | net presence | absent | absent |
| | texture: soft, firm or hard | firm | hard |
| | thickness at medial | 2 mm | 4 mm |
| | primary color (at edible maturity) | buff-yellow | cream |
| | RHS color chart value | 158B | 157B |
| | mottling color (at edible maturity) | buff-yellow | cream |
| | RHS color chart value | 158A | 157C |
| | primary color (at full maturity) | buff-yellow | cream |
| | RHS color chart value | 158B | 157B |
| | mottling color (at full maturity) | buff-yellow | cream |
| | RHS color chart value | 158A | 157C |

*These are typical values. Values may vary due to environment. Other values that are substantially equivalent are also within the scope of the invention.

### Example 8

### Ethylene-Mediated Abscission

Cucumis melo var. inodorus fruits produce very little ethylene during late maturity, after the point where the melons would be harvested in a commercial setting. These ethylene levels increase very gradually over time and do not reach the levels observed in the “climacteric ripening” melon fruits. Climacteric ripening is characterized by the rapid, autocatalytic production of ethylene, and is accompanied by several ethylene mediated physiological and biochemical events such as flesh softening, aroma production, rapid rind color change and abscission from the vine. The autocatalytic production of ethylene manifests as exponentially increasing concentrations of ethylene over time in the cavity of the melon, generally progressing from negligible to a maximum over just a few days. The absolute magnitude of the peak level of ethylene varies among climacteric melon varieties; however the rapid induction of ethylene biosynthesis is characteristic of such lines.

The involvement of ethylene in the harvest maturity indicator trait of Cucumis melo var. inodorus plants was examined in fruit grown in Woodland, Calif. in the summer. Hybrid SVR03968074 and two conventional honeydew lines, Haley and TAM Dew, were planted in a completely randomized design in a greenhouse. The plants were hand pollinated, and the date of pollination was marked. Beginning at 31 days post pollination, three randomly selected fruits of each line were harvested and analyzed as described below. Computer-generated randomization of fruits to be harvested on each day was generated based upon balanced sampling of target days post-pollination of fruit within each variety. Following the initiation of sampling, three fruits were harvested on each of three days a week, until fruit slipped from the vine or until the plants were dead. During the course of the experiment, the fruit of Haley began to form an abscission layer; however this was over three weeks past the time of a commercial harvest for this line. TAM Dew fruits did not show any signs of abscission even after the plants were dead.

Data were collected on ethylene levels, flesh firmness, and Brix levels using established protocols. For ethylene, 250 μL of gas sampled from the fruit cavity were analyzed by GC-FID to determine the ethylene content in parts per million. In some instances it was not possible to obtain a cavity gas sample, as melon seed cavities can be variable in size and gas content; additionally the ability to collect ethylene tends to diminish as the fruit flesh softens and liquid is increasingly released from the flesh. Flesh firmness was assessed using the fruit texture analyzer, and Brix data was collected from the homogenate of equatorial slices of flesh tissue using a bench top refractometer. These lab based protocols generally yield numbers slightly lower than those collected in the field due to differences in the portable equipment and flesh sampling methods; however, the correlations between the methods are extremely robust.

The ethylene data revealed a classical climacteric induction of ethylene levels in the slipping hybrid honeydew SVR03968074. This is in contrast to the basal levels of ethylene observed in the conventional honeydew lines Haley and TAM Dew (FIG. 8). Though the trajectory of ethylene levels over time is clearly a rapid autocatalytic type, it should be noted that the peak levels of ethylene observed in this experiment (27 ppm) is lower than the ranges seen in climacteric cantaloupe melons (generally between 50 and 200 ppm). Notably, in spite of this dramatic difference in ripening physiology, the flesh firmness at the time of commercial harvest was substantially similar to the conventional varieties (FIG. 9). The Brix levels were also unaffected by this novel ripening process in honeydew (FIG. 10). Taken together, these data show that the harvest maturity indicator trait is likely the result of introducing a low level climacteric type ripening process into an otherwise non-climacteric background, which created a harvest indicator in a market type previously lacking this tool, enabling the harvest of consistently high quality fruit.

### Seed Deposits

A deposit of 2500 seeds has been made of the Seminis Vegetable Seeds proprietary lines SVR03968106, HDG39-2021AN, HDG39-2022AN, HDG39-2023AN, and SVR03968074, disclosed above and encompassed in the appended claims, with the American Type Culture Collection (ATCC), Manassas, Va. 20110-2209 USA, an International Depositary Authority (IDA) as established under the Budapest Treaty on the International Recognition of the Deposit of Micro-organisms for the Purposes of Patent Procedure.

Representative samples of seed of SVR03968106 were deposited with ATCC on Aug. 6, 2009 under Accession Number PTA-10254. Representative samples of seed of HDG39-2021AN were deposited with ATCC on Aug. 6, 2009 under PTA-10257. Representative samples of seed of HDG39-2022AN were deposited with ATCC on ______ under ______. Representative samples of seed of HDG39-2023AN were deposited with ATCC on Aug. 6, 2009 under PTA-10258. Representative samples of seed of SVR03968074 were deposited with ATCC on Aug. 6, 2009 under PTA-10255.

Upon issuance of a patent, all restrictions upon the deposits will be removed, and the deposits are intended to meet all of the requirements of 37 C.F.R. §1.801-1.809. The deposits will be maintained in the depository for a period of 30 years, or 5 years after the last request, or for the effective life of the patent, whichever is longer, and will be replaced if necessary during that period.

Although the foregoing invention has been described in some detail by way of illustration and example for purposes of clarity and understanding, it will be obvious that certain changes and modifications may be practiced within the scope of the invention, as limited only by the scope of the appended claims.

All references cited herein are hereby expressly incorporated herein by reference.

### REFERENCES

The following references, to the extent that they provide exemplary procedural or other details supplementary to those set forth herein, are specifically incorporated herein by reference:
Stephens, J. M. Melon, honeydew—Cucumis melo L. (Indorus group). 1994. Document HS626, Florida Cooperative Extension Service, University of Florida.
Suslow, T. V. et al. Honeydew melon: Recommendations for maintaining postharvest quality. 2009. www.postharvest.ucdavis.edu/Produce/ProduceFacts/Fruit/honeydew.shtml
USDA. An Economic Assessment Of Honeydew Melons: Executive Summary. 1999. www.rma.usda.gov/pilots/feasible/txt/honeydew.txt
USDA. United States Standards for Grades of Honey Dew and Honey Ball Type Melons. 1997.

1. A Cucumis melo var. inodorus plant of a commercially acceptable variety, wherein the plant produces melons that exhibit separation from the stem at harvest maturity.

2. The plant of claim 1, wherein the plant produces melons comprising a flesh firmness from about 7 PSI to about 14 PSI, when measured by penetrometer with an 11 millimeter probe, at melon separation from the stem.

3. The plant of claim 1, wherein the plant produces melons comprising a Brix content from about 10° Brix to about 17° Brix, when measured at melon separation from the stem.

4. The plant of claim 1, wherein the plant is inbred.

5. The plant of claim 1, wherein the plant is hybrid.

6. A seed of the plant of claim 1.

7. A plant part of the plant of claim 1.

8. The plant part of claim 7, wherein the plant part is selected from the group consisting of a leaf, pollen, an ovule, a fruit, rootstock, a scion, and a cell.

9. The plant part of claim 8, wherein the plant part is a fruit.

10. A tissue culture of regenerable cells of the plant of claim 1.

11. A tissue culture according to claim 10, comprising cells or protoplasts from a plant part selected from the group consisting of embryos, meristems, cotyledons, pollen, leaves, anthers, roots, root tips, pistil, flower, seed and stalks.

12. A Cucumis melo var. inodorus plant regenerated from the tissue culture of claim 11.

13. The plant of claim 1, wherein the plant comprises a trait for separation from the stem at harvest maturity controlled by genetic means for the expression of such a trait found in a plant selected from the group consisting of inbred line HDG39-2021AN, a sample of seed of which has been deposited under ATCC Accession No. PTA-10257; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

14. The plant of claim 1, wherein the plant is selected from the group consisting of inbred line HDG39-2021AN, a sample of seed of which has been deposited under ATCC Accession No. PTA-10257; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

15. The plant of claim 1, wherein the plant is from a melon market class selected from the group consisting of Piel de Sapo, Juan Canary, Earl's Type, Honeydew, Orange flesh honeydew, Hami Melon, Crenshaw and Casaba.

16. A Cucumis melo var. inodorus plant obtainable by crossing a first plant of claim 1 with a second plant, wherein the Cucumis melo var. inodorus plant produces melons that exhibit separation from the stem at harvest maturity.

17. The Cucumis melo var. inodorus of claim 16, wherein the first plant is a plant of a variety selected from the group consisting of inbred line HDG39-2021AN, a sample of seed of which has been deposited under ATCC Accession No. PTA-10257; inbred line HDG39-2023AN, a sample of seed of which has been deposited under ATCC Accession No. PTA-10258; hybrid line SVR03968106, a sample of seed of which has been deposited under ATCC Accession No. PTA-10254; and hybrid line SVR03968074, a sample of seed of which has been deposited under ATCC Accession No. PTA-10255.

18. A Cucumis melo var. inodorus melon having flesh firmness from about 7 PSI to about 14 PSI, when measured by penetrometer with an 11 millimeter probe and no residual stem upon detachment from the plant.

19. The melon of claim 18, wherein the melon comprises a Brix content from about 10° Brix to about 17° Brix, when measured at melon separation from the stem.

20. A method of identifying a Cucumis melo var. inodorus melon at harvest maturity comprising detecting separation of the melon from the stem.

21. The method of claim 20, wherein detecting separation of the melon from the stem comprises mechanical detection.

22. The method of claim 21, wherein mechanical detection comprises detecting a reduction in resistance in melon separation from the stem.

23. The method of claim 20, wherein detecting separation of the melon from the stem comprises visual detection.

24. A method of producing a plant of claim 1 comprising:
(a) crossing a first Cucumis melo var. inodorus plant with a second melon plant capable of being crossed to said first Cucumis melo var. inodorus plant and that exhibits separation of a melon from the stem at harvest maturity;
(b) selecting an F1 progeny that exhibits separation of the melon from the stem at harvest maturity;
(c) crossing the selected F1 progeny with the first Cucumis melo var. inodorus plant to produce a backcross progeny;
(d) selecting a backcross progeny that exhibits separation of the melon from the stem at harvest maturity and comprising the physiological and morphological characteristics of the first Cucumis melo var. inodorus plant; and
(e) repeating steps (c) and (d) three or more times to produce a selected fourth or higher backcross progeny plant that exhibits separation of the melon from the stem at harvest maturity.

25. The method of claim 24, wherein the second melon plant is a plant other than a Cucumis melo var. inodorus plant.

26. The method of claim 25, wherein the second melon plant is a plant of line PI 414723.

27. The method of claim 24, further comprising:
(f) crossing the selected backcross progeny plant with a second Cucumis melo var. inodorus plant to produce seed of a hybrid progeny plant.

28. A method of producing a Cucumis melo var. inodorus seed, comprising crossing a first plant according to claim 1 with itself or a second Cucumis melo var. inodorus plant.

29. The method of claim 28, wherein the first plant is used as female parent in said crossing.

30. The method of claim 28, wherein the first plant is used as a male parent in said crossing.

31. A seed produced by the method of claim 28.

32. A plant produced by growing the seed of claim 31.

33. A method for producing a seed of a plant derived from a plant of claim 1 comprising:
(a) crossing a first plant according to claim 1 with a second plant capable of being crossed to said first plant; and
(b) allowing seed to form.

34. The method of claim 33, further comprising:
(c) crossing a plant grown from said seed with itself or a second plant capable of being crossed to the plant grown from said seed to yield additional seed;
(d) growing said additional seed of step (c) to yield further plants; and
(e) repeating the crossing and growing steps of (c) and (d) to generate at least a first plant further derived from the plant of claim 1.

35. The method of claim 34, wherein the second plant is of an inbred Cucumis melo var. inodorus line.

36. The method of claim 34, further comprising:
(f) crossing the at least a first plant further derived from the plant of claim 1 with a second plant capable of being crossed to said at least a first plant further derived from the plant of claim 1 to produce seed of a hybrid progeny plant.

37. A method of vegetatively propagating the plant of claim 1 comprising:
(a) obtaining tissue capable of being propagated from a plant according to claim 1;
(b) cultivating said tissue to obtain proliferated shoots; and
(c) rooting said proliferated shoots to obtain rooted plantlets.

38. The method of claim 37, further comprising growing plants from said rooted plantlets.

39. A method of introducing a desired trait into a Cucumis melo var. inodorus line comprising:
(a) crossing a plant of claim 1 with a second Cucumis melo var. inodorus plant that comprises a desired trait to produce F1 progeny; and
(b) selecting an F1 progeny that comprises the desired trait.

40. The method of claim 39, further comprising:
(c) crossing the selected F1 progeny with a plant of claim 1 to produce a backcross progeny;
(d) selecting backcross progeny comprising the desired trait and physiological and morphological characteristics of the Cucumis melo var. inodorus plant of claim 1; and
(e) repeating steps (c) and (d) three or more times to produce selected fourth or higher backcross progeny that comprise the desired trait.

41. A Cucumis melo var. inodorus plant produced by the method of claim 39.

42. A method of producing a plant comprising an added desired trait, the method comprising introducing a transgene conferring the desired trait into a plant of claim 1.

43. A method of determining the genotype of the plant of claim 1 comprising obtaining a sample of nucleic acids from said plant and detecting in said nucleic acids a plurality of polymorphisms.

44. The method of claim 43, further comprising the step of storing the results of detecting the plurality of polymorphisms on a computer readable medium.

45. A computer readable medium produced by the method of claim 44.

46. A method of determining the genotype of the plant of claim 1, comprising obtaining a sample of nucleic acids from said plant and determining the nucleic acid sequence of at least one locus.

47. A method of producing melons comprising:
(a) obtaining the plant of claim 1, wherein the plant has been cultivated to maturity; and
(b) collecting melons from the plant.

48. A melon plant comprising at least a first set of the chromosomes of melon line HDG39-2022AN, a sample of seed of said lines having been deposited under ATCC Accession Number PTA-11280.

49. A seed comprising at least a first set of the chromosomes of melon line HDG39-2022AN, a sample of seed of said lines having been deposited under ATCC Accession Number PTA-11280.

50. The plant of claim 48, which is inbred.

51. The plant of claim 48, which is hybrid.

52. The plant of claim 48, wherein the plant is a plant of line HDG39-2022AN.

53. A plant part of the plant of claim 48.

54. The plant part of claim 53, further defined as a leaf, a ovule, pollen, a fruit, or a cell.

55. A plant part of the plant of claim 48.

56. The plant part of claim 55, further defined as a leaf, a ovule, pollen, a fruit, or a cell.

57. A tissue culture of regenerable cells of the plant of claim 48.

58. A method of introducing a desired trait into a melon line comprising:
(a) crossing a plant of line HDG39-2022AN with a second melon plant that comprises a desired trait to produce F1 progeny, a sample of seed of said line having been deposited under ATCC Accession Number PTA-11280;
(b) selecting an F1 progeny that comprises the desired trait;
(c) backcrossing the selected F1 progeny with a plant of line HDG39-2022AN to produce backcross progeny;
(d) selecting backcross progeny comprising the desired trait and the physiological and morphological characteristic of melon line HDG39-2022AN; and
(e) repeating steps (c) and (d) three or more times to produce selected fourth or higher backcross progeny that comprise the desired trait.

59. A melon plant produced by the method of claim 57.

60. A method of producing a plant comprising an added trait, the method comprising introducing a transgene conferring the trait into a plant of line HDG39-2022AN, a sample of seed of said line having been deposited under ATCC Accession Number PTA-11280.

61. A plant produced by the method of claim 59.

62. A method for producing a seed of a plant derived from line HDG39-2022AN comprising the steps of:
(a) crossing a melon plant of line HDG39-2022AN with itself or a second melon plant; a sample of seed of said line having been deposited under ATCC Accession Number PTA-11280; and
(b) allowing seed of a line HDG39-2022AN-derived melon plant to form.

63. The method of claim 62, further comprising the steps of:
(c) selfing a plant grown from said line HDG39-2022AN-derived melon seed to yield additional HDG39-2022AN-derived melon seed;
(d) growing said additional line HDG39-2022AN-derived melon seed of step (c) to yield additional line HDG39-2022AN-derived melon plants; and
(e) repeating the crossing and growing steps of (c) and (d) to generate at least a first line HDG39-2022AN-derived melon plant.

64. The method of claim 63, further comprising:
(f) crossing the further line HDG39-2022AN-derived melon plant with a second melon plant to produce seed of a hybrid progeny plant.
